(19) United States
(12) Patent Application Publication
(10) Pub. No.: US 2008/0015202 A1
(43) Pub. Date: Jan. 17, 2008

(54) Thiazole Derivatives Having Vap-1 Inhibitory Activity

(75) Inventors: Takayuki Inoue, Tokyo (JP); Takashi Tojo, Tokyo (JP); Masataka Morita, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Astellas Pharma Inc., Chuo-ku, Tokyo (JP)

(21) Appl. No.: 11/574,946

(22) PCT Filed: Sep. 8, 2005
(86) PCT No.: PCT/JP05/16984

§ 371 Date: Mar. 8, 2007

(30) Foreign Application Priority Data
Sep. 9, 2004 (AU) 2004905183

(51) Int. Cl. A61K 31/426; A61K 31/445; A61K 31/497; A61P 27/02; C07D 411/00

(52) U.S. Cl. 514/254.04; 514/326; 514/370; 544/369; 546/209; 548/190

(57) ABSTRACT

A compound of the formula (I):
U-V-W-X-Y-Z (I) wherein U is lower alkyl;
V is —CONH— or —NR¹CO— wherein R¹ is a hydrogen or lower alkyl; W is a bond or lower alkylene; X is a bivalent residue derived from optionally substituted thiazole; Y is a bond or lower alkylene; and
   Z is a group of the formula: 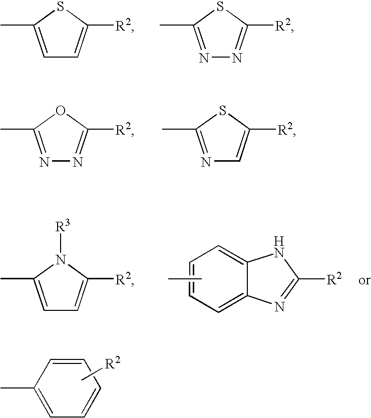
   wherein R² is a group of the formula: -A-B-D-E-F-G wherein A is a bond or lower alkylene;
      B is a bond, —NH— or 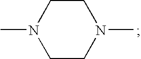 D is a bond, —CS— or —CO—; E is a bond or —NH—;
      F is a bond, —CO—, —O— or —SO₂—; and
         G is lower alkyl, optionally protected amino, —OH, phenyl, 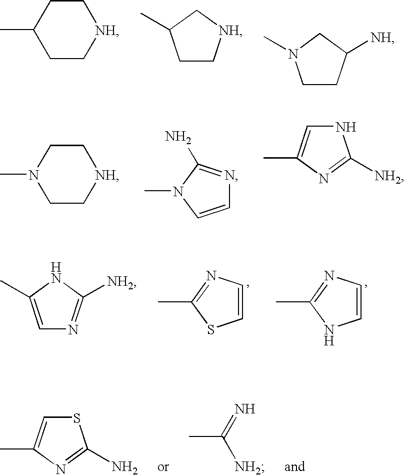
   R³ is lower alkyl, provided that
      when Z is a group of the formula: 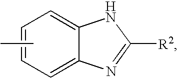 then G should not be amino,
      when Z is a group of the formula: 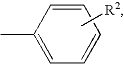
      then G should not be 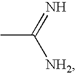
      when Z is a group of the formula: 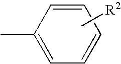 and G is optionally protected amino, then D should be —CS—, or then A should be lower alkylene, B or E should be —NH— and F should be —CO—; or a pharmaceutically acceptable salt thereof useful as a vascular adhesion protein-1 (VAP-1) inhibitor as well as a pharmaceutical composition and a method for preventing or treating a VAP-1 associated disease, especially macular edema, which method includes administering an effective amount of the compound or a pharmaceutically acceptable salt thereof to a subject, and the like.

### TECHNICAL FIELD

The present invention relates to a compound or a pharmaceutically acceptable salt thereof useful as a vascular adhesion protein-1 inhibitor, a pharmaceutical composition comprising the compound or salt thereof as an active ingredient, a method for preventing or treating a vascular adhesion protein-1 associated disease, especially macular edema, use of the compound, salt thereof or composition, and the like.

### BACKGROUND ART

Vascular adhesion protein-1 (hereinafter to be abbreviated as VAP-1) is an amine oxidase (semicarbazide sensitive amine oxidase, SSAO) which is abundant in human plasma, and shows remarkably increased expression in vascular endothelium and vascular smooth muscle of the inflammatory region. While the physiological role of VAP-1 has not been clarified until recently, VAP-1 gene was cloned in 1998, and VAP-1 has been reported to be a membrane protein that regulates rolling and migration of lymphocyte and NK cell as an adhesion molecule under regulation of expression by inflammatory cytokine. Although the amine to be a substrate is unknown, it is considered to be methylamine generated in any part of living organisms. It is also known that hydrogen peroxide and aldehydes produced due to the amine oxidase activity in the molecule are important factors of adhesion activity.

A recent report has documented that VAP-1 enzyme activity in plasma increases in diabetic patients, whether type I or type II, and the increase is particularly remarkable in diabetic patients suffering from retinopathy complications (Diabetologia, 42 (1999) 233-237 and Diabetic Medicine, 16 (1999) 514-521).

In addition, it has been reported that VAP-1 is associated with the following diseases:

(1) cirrhosis, essential stabilized hypertension, diabetes, and arthrosis (see JP-A-61-239891 and U.S. Pat. No. 4,888,283);

(2) endothelium damage (in diabetes, atherosclerosis and hypertension), a cardiovascular disorder associated with diabetes and uremia, pain associated with gout and arthritis, and retinopathy (in diabetes patients) (see WO 93/23023);

(3) a (connective tissue) inflammatory disease or condition (rheumatoid arthritis, ankylosing spondylitis, psoriatic arthritis and osteoarthritis or degenerative joint disease, Reiter's syndrome, Sjögren's syndrome, Behçet's syndrome, relapsing polychondritis, systemic lupus erythematosus, discoid lupus erythematosus, systemic sclerosis, eosinophilic fasciitis, polymyositis, dermatomyositis, polymyalgia rheumatica, vasculitis, temporal arteritis, polyarteritis nodosa, Wegener's granulomatosis, mixed connective tissue disease, and juvenile rheumatoid arthritis); a gastrointestinal inflammatory disease or condition [Crohn's disease, ulcerative colitis, irritable bowel syndrome (spastic colon), fibrotic conditions of the liver, inflammation of the oral mucosa (stomatitis), and recurrent aphtous stomatitis]; a central nervous system inflammatory disease or condition (multiple sclerosis, Alzheimer's disease, and ischemia-reperfusion injury associated with ischemic stroke); a pulmonary inflammatory disease or condition (asthma, adult respiratory distress syndrome, and chronic obstructive pulmonary disease); a (chronic) skin inflammatory disease or condition (psoriasis, allergic lesions, lichen planus, pityriasis rosea, contact dermatitis, atopic dermatitis, and pityriasis rubra pilaris); a disease related to carbohydrate metabolism (diabetes and complications from diabetes) including microvascular and macrovascular disease (atherosclerosis, vascular retinopathies, retinopathy, nephropathy, nephrotic syndrome and neuropathy (polyneuropathy, mononeuropathies and autonomic neuropathy), foot ulcers, joint problems, and increased risk of infection); a disease related to aberrations in adipocyte differentiation or function or smooth muscle cell function (atherosclerosis and obesity); a vascular disease [atheromatous ateriosclerosis, nonatheromatous ateriosclerosis, ischemic heart disease including myocardial infarction and peripheral arterial occlusion, Raynaud's disease and phenomenon, and thromboangiitis obliterans (Buerger's disease)]; chronic arthritis; inflammatory bowel diseases; skin dermatoses (see WO 02/02090, WO 02/02541 and US patent application publication No. 2002/0173521 A1);

(4) diabetes mellitus (see WO 02/38152);

(5) SSAO-mediated complication [diabetes (insulin dependent diabetes mellitus (IDDM) and non-insulin dependent diabetes mellitus (NIDDM)) and vascular complication (heart attack, angina, strokes, amputations, blindness and renal failure)] (see WO 02/38153);

(6) hepatitis, transplantation, and the like.

Under the present circumstances, a drug treatment or prophylaxis of the above diseases has been demanded.

In addition, macular edema is a common ocular abnormality resulting from vast etiology and characterized by perturbation of the integrity of the blood-retinal barrier of the perifoveal capillaries and the optic nerve head. Macular edema is known to include diabetic and non-diabetic macular edema. Macular edema as a diabetic complication is a disease state that can occur in any stage of diabetic retinopathy, emerges before the onset of neovascularization and causes serious visual disorders. Macular area is a highly evolved part in retina and plays a key role in controlling the eyesight. Once the macular area suffers from edema, how mild the change may be, it causes a significant failure of eyesight, and when left unattended, the edema causes irreversible changes of macular tissue, and it is considered to encourage progress of retinopathy.

At present, for macular edema, laser beam photocoagulation and vitreous surgery have been tried as a symptomatic therapy. However, irradiation of laser on the macular area is not easy and unnecessary laser treatments may produce side effects (e.g., possible encouragement of edema by causing inflammation). The vitreous surgery is considered to provide efficacy in 70 percent of macular edema, but physical and economical burden on patients is high, and the incidence of recurrence is also high. These treatment methods are not usually employed in the initial stage of macular edema, particularly so in the stages where the decrease of vision is comparatively small. Accordingly, a drug treatment comparatively easily applicable from the early stages of the disease has been also demanded under the present circumstances.

### DISCLOSURE OF INVENTION

The present inventors have intensively worked on the problem of the drug treatment of a VAP-1 associated disease and found that a VAP-1 inhibitor of the present invention is useful for the prophylaxis or treatment of the disease, particularly macular edema, and completed the present invention. Thus, the present invention provides the following.

[1] A compound of the formula (I) [hereinafter sometimes referred to as Compound (1) or VAP-1 inhibitor]:
U-V-W-X-Y-Z (I)
wherein
U is lower alkyl;
V is —CONH— or —NR¹CO— wherein R¹ is a hydrogen or lower alkyl;
W is a bond or lower alkylene;
X is a bivalent residue derived from optionally substituted thiazole;
Y is a bond or lower alkylene; and
Z is a group of the formula: 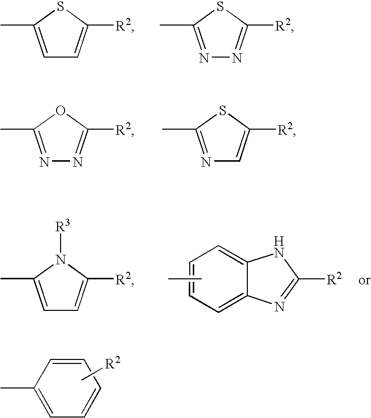
   wherein R² is a group of the formula: -A-B-D-E-F-G
      wherein
         A is a bond or lower alkylene;
         B is a bond, —NH— or 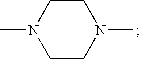
         D is a bond, —CS— or —CO—;
         E is a bond or —NH—;
         F is a bond, —CO—, —O— or —SO₂—; and
         G is lower alkyl, optionally protected amino, —OH, phenyl, 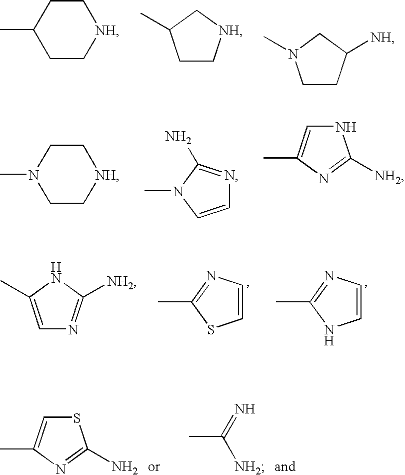
   R³ is lower alkyl,
      provided that
      when Z is a group of the formula: 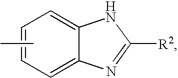
      then G should not be amino,
      when Z is a group of the formula: 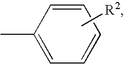
      then G should not be 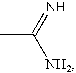
      when Z is a group of the formula: 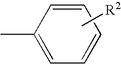
      and G is optionally protected amino,
   then D should be —CS—, or
   then A should be lower alkylene,
   B or E should be —NH— and F should be —CO—;
      or a pharmaceutically acceptable salt thereof.
      [2] The compound of [1], wherein the compound is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide,
   or a pharmaceutically acceptable salt thereof.
   [3] The compound of [1] or a pharmaceutically acceptable salt thereof for use as a medicament.
   [4] A pharmaceutical composition, which comprises, as an active ingredient, the compound of [1] or a pharmaceutically acceptable salt thereof.
   [5] The pharmaceutical composition of [4], wherein the compound of the formula (I) is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide.
   [6] Use of the compound of [1] or a pharmaceutically acceptable salt thereof for preparing a medicament as a VAP-1 inhibitor.
   [7] The use of [6], wherein the compound is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide,
   or a pharmaceutically acceptable salt thereof.
   [8] Use of the compound of [1] or a pharmaceutically acceptable salt thereof for preparing a medicament for the prophylaxis or treatment of a VAP-1 associated disease.
   [9] The use of [8], wherein said VAP-1 associated disease is selected from the group consisting of cirrhosis, essential stabilized hypertension, diabetes, arthrosis, endothelium damage (in diabetes, atherosclerosis and hypertension), a cardiovascular disorder associated with diabetes and uremia, pain associated with gout and arthritis, retinopathy (in diabetes patients), a (connective tissue) inflammatory disease or condition (rheumatoid arthritis, ankylosing spondylitis, psoriatic arthritis, osteoarthritis, degenerative joint disease, Reiter's syndrome, Sjögren's syndrome, Behçet's syndrome, relapsing polychondritis, systemic lupus erythematosus, discoid lupus erythematosus, systemic sclerosis, eosinophilic fasciitis, polymyositis, dermatomyositis, polymyalgia rheumatica, vasculitis, temporal arteritis, polyarteritis nodosa, Wegener's granulomatosis, mixed connective tissue disease, and juvenile rheumatoid arthritis), a gastrointestinal inflammatory disease or condition [Crohn's disease, ulcerative colitis, irritable bowel syndrome (spastic colon), fibrotic conditions of the liver, inflammation of the oral mucosa (stomatitis), and recurrent aphtous stomatitis], a central nervous system inflammatory disease or condition (multiple sclerosis, Alzheimer's disease, and ischemia-reperfusion injury associated with ischemic stroke), a pulmonary inflammatory disease or condition (asthma, adult respiratory distress syndrome, and chronic obstructive pulmonary disease), a (chronic) skin inflammatory disease or condition (psoriasis, allergic lesions, lichen planus, pityriasis rosea, contact dermatitis, atopic dermatitis, and pityriasis rubra pilaris), a disease related to carbohydrate metabolism (diabetes and complications from diabetes) including microvascular and macrovascular disease (atherosclerosis, vascular retinopathies, retinopathy, nephropathy, nephrotic syndrome and neuropathy (polyneuropathy, mononeuropathies and autonomic neuropathy), foot ulcers, joint problems, and increased risk of infection), a disease related to aberrations in adipocyte differentiation or function or smooth muscle cell function (atherosclerosis and obesity), a vascular disease [atheromatous ateriosclerosis, nonatheromatous ateriosclerosis, ischemic heart disease including myocardial infarction and peripheral arterial occlusion, Raynaud's disease and phenomenon, and thromboangiitis obliterans (Buerger's disease)], chronic arthritis, inflammatory bowel diseases, skin dermatoses, diabetes mellitus, SSAO-mediated complication [diabetes (insulin dependent diabetes mellitus (IDDM) and non-insulin dependent diabetes mellitus (NIDDM)) and vascular complication (heart attack, angina, strokes, amputations, blindness, and renal failure)], macular edema (diabetic and non-diabetic macular edema), hepatitis, and transplantation.
   [10] The use of [9], wherein said VAP-1 associated disease is macular edema.
   [11] The use of [10], wherein said macular edema is diabetic macular edema.
   [12] The use of [10], wherein said macular edema is non-diabetic macular edema.
   [13] A VAP-1 inhibitor, which comprises the compound of [1] or a pharmaceutically acceptable salt thereof.
   [14] A method for preventing or treating macular edema, which method comprises administering to a subject in need thereof a VAP-1 inhibitor in an amount sufficient to treat said subject for macular edema.
   [15] The method of [14], wherein the VAP-1 inhibitor is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide,
   or a pharmaceutically acceptable salt thereof.
   [16] A method for preventing or treating a VAP-1 associated disease, which method comprises administering an effective amount of the compound of [1] or a pharmaceutically acceptable salt thereof to a subject in need thereof.
   [17] The method of [16], wherein said VAP-1 associated disease is selected from the group consisting of cirrhosis, essential stabilized hypertension, diabetes, arthrosis, endothelium damage (in diabetes, atherosclerosis and hypertension), a cardiovascular disorder associated with diabetes and uremia, pain associated with gout and arthritis, retinopathy (in diabetes patients), a (connective tissue) inflammatory disease or condition (rheumatoid arthritis, ankylosing spondylitis, psoriatic arthritis, osteoarthritis, degenerative joint disease, Reiter's syndrome, Sjögren's syndrome, Behçet's syndrome, relapsing polychondritis, systemic lupus erythematosus, discoid lupus erythematosus, systemic sclerosis, eosinophilic fasciitis, polymyositis, dermatomyositis, polymyalgia rheumatica, vasculitis, temporal arteritis, polyarteritis nodosa, Wegener's granulomatosis, mixed connective tissue disease, and juvenile rheumatoid arthritis), a gastrointestinal inflammatory disease or condition [Crohn's disease, ulcerative colitis, irritable bowel syndrome (spastic colon), fibrotic conditions of the liver, inflammation of the oral mucosa (stomatitis), and recurrent aphtous stomatitis], a central nervous system inflammatory disease or condition (multiple sclerosis, Alzheimer's disease, and ischemia-reperfusion injury associated with ischemic stroke), a pulmonary inflammatory disease or condition (asthma, adult respiratory distress syndrome, and chronic obstructive pulmonary disease), a (chronic) skin inflammatory disease or condition (psoriasis, allergic lesions, lichen planus, pityriasis rosea, contact dermatitis, atopic dermatitis, and pityriasis rubra pilaris), a disease related to carbohydrate metabolism (diabetes and complications from diabetes) including microvascular and macrovascular disease (atherosclerosis, vascular retinopathies, retinopathy, nephropathy, nephrotic syndrome and neuropathy (polyneuropathy, mononeuropathies and autonomic neuropathy), foot ulcers, joint problems, and increased risk of infection), a disease related to aberrations in adipocyte differentiation or function or smooth muscle cell function (atherosclerosis and obesity), a vascular disease [atheromatous ateriosclerosis, nonatheromatous ateriosclerosis, ischemic heart disease including myocardial infarction and peripheral arterial occlusion, Raynaud's disease and phenomenon, and thromboangiitis obliterans (Buerger's disease)], chronic arthritis, inflammatory bowel diseases, skin dermatoses, diabetes mellitus, SSAO-mediated complication [diabetes (insulin dependent diabetes mellitus (IDDM) and non-insulin dependent diabetes mellitus (NIDDM)) and vascular complication (heart attack, angina, strokes, amputations, blindness and renal failure)], macular edema (diabetic and non-diabetic macular edema), hepatitis, and transplantation.
   [18] The method of [17], wherein said VAP-1 associated disease is macular edema.
   [19] The method of [18], wherein said macular edema is diabetic macular edema.
   [20] The method of [18], wherein said macular edema is non-diabetic macular edema.

### DETAILED DESCRIPTION OF THE INVENTION

The present invention is predicated on the discovery that an inhibitor for vascular adhesion protein-1 (VAP-1; also referred to as semicarbazide sensitive amine oxidase (SSAO) or copper-containing amine oxidase) is effective in treating or ameliorating VAP-1 associated diseases, especially macular edema, and the like. Accordingly, the present invention provides Compound (1) and a pharmaceutically acceptable salt thereof useful as a VAP-1 inhibitor as well as a pharmaceutical composition and a method for preventing or treating a VAP-1 associated disease, and the like.

In the above and subsequent descriptions of the present specification, suitable examples and illustration of the various definitions to be included within the scope of the invention are explained in detail as follows.

Suitable “halogen” includes fluorine, chlorine, bromine and iodine.

The term “lower” is used to intend a group having 1 to 6, preferably 1 to 4, carbon atom(s), unless otherwise provided.

Suitable “lower alkyl” includes straight or branched alkyl having 1 to 6 carbon atom(s), such as methyl, ethyl, propyl, isopropyl, butyl, isobutyl, sec-butyl, tert-butyl, pentyl, tert-pentyl and hexyl, in which more preferred one is C₁-C₄ alkyl.

Suitable “lower alkylene” includes straight or branched alkylene having 1 to 6 carbon atom(s), such as methylene, ethylene, trimethylene, tetramethylene, propylene, ethylidene and propylidene, in which more preferred one is C₁-C₄ alkylene.

Suitable “lower alkenylene” includes straight or branched alkenylene having 2 to 6 carbon atom(s), such as —CH═CH—, —CH₂—CH═CH—, —CH₂—CH═CH—CH₂—, —CH₂—CH₂—CH═CH—, —CH═CH—CH═CH—, —CH═CH—CH₂—CH₂—CH₂—, —CH═CH—CH═CH—CH₂—CH₂— and —CH═CH—CH═CH—CH═CH—, in which more preferred one is C₂-C₄ alkenylene.

The above lower alkenylene may be each in E or Z form. Thus, one of ordinary skill in the art will recognize that the lower alkenylene includes all E, Z-structures when it has 2 or more double bonds.

Suitable “aryl” includes C₆-C₁₀ aryl such as phenyl and naphthyl, in which more preferred one is phenyl. The “aryl” may be substituted by 1 to 3 substituent(s) and the substitution sites are not particularly limited.

Suitable “aralkyl” includes aralkyl wherein the aryl moiety has 6 to 10 carbon atoms [i.e. the aryl moiety is C₆-C₁₀ aryl of the above “aryl”] and the alkyl moiety has 1 to 6 carbon atom(s) [i.e. the alkyl moiety is C₁-C₆ alkyl of the above “lower alkyl”], such as benzyl, phenethyl, 1-naphthylmethyl, 2-naphthylmethyl, 3-phenylpropyl, 4-phenylbutyl and 5-phenylpentyl.

The “optionally protected amino” means that an amino group may be protected with a suitable protecting group according to a method known per se, such as the methods described in Protective Groups in Organic Synthesis, published by John Wiley and Sons (1980), and the like. The suitable “protecting group” includes tert-butoxycarbonyl (i.e. Boc), an acyl group as mentioned below, substituted or unsubstituted aryl(lower)alkylidene [e.g., benzylidene, hydroxybenzylidene, etc.], aryl(lower)alkyl such as mono-, di- or triphenyl-(lower)alkyl [e.g., benzyl, phenethyl, benzhydryl, trityl, etc.], and the like.

Suitable “optionally protected amino” includes amino and tert-butoxycarbonylamino (i.e. —NHBoc).

Suitable “heterocycle” includes “aromatic heterocycle” and “non-aromatic heterocycle”.

Suitable “aromatic heterocycle” includes 5 to 10-membered aromatic heterocycle containing 1 to 3 heteroatom(s) selected from nitrogen, oxygen and sulfur atoms besides carbon atom(s), and includes, for example, thiophene, furan, pyrrole, imidazole, pyrazole, thiazole, isothiazole, oxazole, isoxazole, pyridine, pyridazine, pyrimidine, pyrazine and the like.

Suitable “non-aromatic heterocycle” includes 5 to 10-membered non-aromatic heterocycle containing 1 to 3 heteroatom(s) selected from nitrogen, oxygen and sulfur atoms besides carbon atom(s), and includes, for example, pyrrolidine, imidazoline, pyrazolidine, pyrazoline, piperidine, piperazine, morpholine, thiomorpholine, dioxolan, oxazolidine, thiazolidine, triazolidine and the like.

Suitable “acyl” includes acyl having 1 to 20 carbon atom(s), such as formyl, alkylcarbonyl, arylcarbonyl, alkoxycarbonyl and aralkyloxycarbonyl.

Suitable “alkylcarbonyl” includes alkylcarbonyl wherein the alkyl moiety has 1 to 6 carbon atom(s) [i.e. the alkyl moiety is C₁-C₆ alkyl of the above “lower alkyl”], such as acetyl, propionyl, butyryl, isobutyryl, valeryl, isovaleryl, pivaloyl, hexanoyl and heptanoyl, in which more preferred one is C₁-C₄ alkyl-carbonyl.

Suitable “arylcarbonyl” includes arylcarbonyl wherein the aryl moiety has 6 to 10 carbon atom(s) [i.e. the aryl moiety is C₆-C₁₀ aryl of the above “aryl”], such as benzoyl and naphthoyl.

Suitable “alkoxycarbonyl” includes alkoxycarbonyl wherein the alkoxy moiety has 1 to 6 carbon atom(s), such as methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, tert-pentyloxycarbonyl and hexyloxycarbonyl, in which more preferred one is alkoxycarbonyl wherein the alkoxy moiety has 1 to 4 carbon atom(s).

Suitable “aralkyloxycarbonyl” includes aralkyloxycarbonyl wherein the aryl moiety has 6 to 10 carbon atoms [i.e. the aryl moiety is C₆-C₁₀ aryl of the above “aryl”] and the alkyl moiety has 1 to 6 carbon atom(s) [i.e. the alkyl moiety is C₁-C₆ alkyl of the above “lower alkyl”], such as benzyloxycarbonyl, phenethyloxycarbonyl, 1-naphthylmethyloxycarbonyl, 2-naphthylmethyloxycarbonyl, 3-phenylpropyloxycarbonyl, 4-phenylbutyloxycarbonyl and 5-phenylpentyloxycarbonyl.

Suitable “bivalent residue derived from thiazole” of the “bivalent residue derived from optionally substituted thiazole” includes

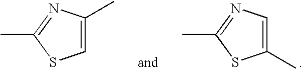

The “thiazole” may have 1 to 3 substituent(s) and the substitution sites are not particularly limited.

Suitable “substituent” of the above “optionally substituted thiazole” includes, for example,

(1) halogen;

(2) alkoxycarbonyl such as ethoxycarbonyl;

(3) optionally substituted aryl, the substitution sites are not particularly limited, such as phenyl and 4-(methylsulfonyl)phenyl;

(4) a group of the formula: —CONR^{a1}R^{a2} wherein R^{a1} and R^{a2} are independently hydrogen, lower alkyl, aryl or aralkyl, such as N-methylaminocarbonyl, N-phenylaminocarbonyl, N,N-dimethylaminocarbonyl and N-benzylaminocarbonyl;

(5) a group of the formula: —CONH—(CH₂)ₖ-aryl wherein k is an integer of 0 to 6; the aryl may have 1 to 5 substituent(s) selected from the group consisting of —NO₂, —SO₂-(lower alkyl), —CF₃ and —O-aryl, and the substitution sites are not particularly limited;

(6) a group of the formula: —CONH—(CH₂)ₘ-heterocycle wherein m is an integer of 0 to 6;

(7) a group of the formula: —CO-heterocycle wherein the heterocycle may have 1 to 5 substituent(s) selected from the group consisting of —CO-(lower alkyl), —CO—O-(lower alkyl), —SO₂-(lower alkyl), oxo (i.e. ═O) and a group of the formula: —CONR^{b1}R^{b2} wherein R^{b1} and R^{b2} are independently hydrogen, lower alkyl, aryl or aralkyl, and the substitution sites are not particularly limited;

(8) a group of the formula: —(CH₂)ₙ-aryl wherein n is an integer of 1 to 6; the aryl may have 1 to 5 substituent(s) selected from the group consisting of —S-(lower alkyl), —SO₂-(lower alkyl), —CO₂-(lower alkyl), —NHCO—O-(lower alkyl) and a group of the formula: —CONR^{c1}R^{c2} wherein R^{c1} and R^{c2} are independently hydrogen, lower alkyl, aryl or aralkyl, and the substitution sites are not particularly limited;

(9) a group of the formula: —(CH₂)ₚ-heterocycle wherein p is an integer of 0 to 6; the heterocycle may have 1 to 5 substituent(s) selected from the group consisting of oxo (i.e. ═O); —CO-(lower alkyl); —CO—O-(lower alkyl); —SO₂-(lower alkyl); —CO-(heterocycle) wherein the heterocycle may have 1 to 5 substituent(s) selected from the group consisting of lower alkyl and halogen, and the substitution sites are not particularly limited; and a group of the formula: —CONR^{d1}R^{d2} wherein R^{d1} and R^{d2} are independently hydrogen, lower alkyl, aryl or aralkyl, and the substitution sites are not particularly limited;

(10) a group of the formula: —(CH₂)ᵣ—NR^{e1}R^{e2}

wherein r is an integer of 0 to 6; R^{e1} and R^{e2} are independently hydrogen, acyl, lower alkyl, aryl or aralkyl, and the lower alkyl may have 1 to 5 substituent(s) selected from the group consisting of a group of the formula: —CONR^{f1}R^{f2} wherein R^{f1} and R^{f2} are independently hydrogen, lower alkyl, aryl or aralkyl, and the substitution sites are not particularly limited;

(11) a group of the formula:
—CON(H or lower alkyl)-(CHR^{g})ₛ-T
wherein s is an integer of 0 to 6; R^{g} is hydrogen, aralkyl, or lower alkyl which may be substituted by 1 to 3 substituent(s) selected from the group consisting of —OH and —CONH₂ and the substitution sites are not particularly limited; and T is hydrogen; a group of the formula: —CONR^{h1}R^{h2} wherein R^{h1} and R^{h2} are independently hydrogen, lower alkyl, aryl or aralkyl; —NH—CO—Rⁱ wherein Rⁱ is lower alkyl or aralkyl; —NH—SO₂-(lower alkyl); —SO₂-(lower alkyl); -heterocycle which may have 1 to 3 substituent(s) such as oxo (i.e. ═O), and the substitution sites are not particularly limited; or —CO— (heterocycle); and

(12) a group of the formula: —(CH₂)ₜ—CO—NR^{j1}R^{j2}

wherein t is an integer of 1 to 6; R^{j1} and R^{j2} are independently hydrogen, lower alkyl, aryl or aralkyl.

The substitution site on the aryl or heterocycle is any suitable position thereof, and is not particularly limited.

The “bivalent residue derived from optionally substituted thiazole” is preferably

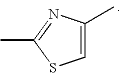

The substitution site of R² on the phenyl in Compound (I) is not particularly limited.

When Z is a group of the formula: 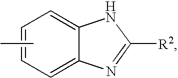
the substitution site on the group is not particularly limited. 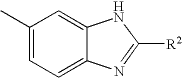
is particularly preferable.

Any nitrogen atom in the amino (i.e. —NH₂), imino (i.e. ═NH or —NH—) or the like in Compound (I) may be protected according to the methods known to one of ordinary skill in the art, such as the methods described in Protective Groups in Organic Synthesis, published by John Wiley and Sons (1980), and the like.

When Compound (I) has an asymmetric carbon atom in the structure, one of ordinary skill in the art will recognize that Compound (I) includes all stereoisomers.

The “vascular adhesion protein-1 (VAP-1) associated disease” comprises a disease selected from the group consisting of cirrhosis, essential stabilized hypertension, diabetes, arthrosis; endothelium damage (in diabetes, atherosclerosis and hypertension), a cardiovascular disorder associated with diabetes and uremia, pain associated with gout and arthritis, retinopathy (in diabetes patients); a (connective tissue) inflammatory disease or condition (rheumatoid arthritis, ankylosing spondylitis, psoriatic arthritis and osteoarthritis, degenerative joint disease, Reiter's syndrome, Sjögren's syndrome, Behçet's syndrome, relapsing polychondritis, systemic lupus erythematosus, discoid lupus erythematosus, systemic sclerosis, eosinophilic fasciitis, polymyositis, dermatomyositis, polymyalgia rheumatica, vasculitis, temporal arteritis, polyarteritis nodosa, Wegener's granulomatosis, mixed connective tissue disease, and juvenile rheumatoid arthritis); a gastrointestinal inflammatory disease or condition [Crohn's disease, ulcerative colitis, irritable bowel syndrome (spastic colon), fibrotic conditions of the liver, inflammation of the oral mucosa (stomatitis), and recurrent aphtous stomatitis]; a central nervous system inflammatory disease or condition (multiple sclerosis, Alzheimer's disease, and ischemia-reperfusion injury associated with ischemic stroke); a pulmonary inflammatory disease or condition (asthma, adult respiratory distress syndrome, and chronic obstructive pulmonary disease); a (chronic) skin inflammatory disease or condition (psoriasis, allergic lesions, lichen planus, pityriasis rosea, contact dermatitis, atopic dermatitis, and pityriasis rubra pilaris); a disease related to carbohydrate metabolism (diabetes and complications from diabetes) including microvascular and macrovascular disease (atherosclerosis, vascular retinopathies, retinopathy, nephropathy, nephrotic syndrome and neuropathy (polyneuropathy, mononeuropathies and autonomic neuropathy), foot ulcers, joint problems, and increased risk of infection); a disease related to aberrations in adipocyte differentiation or function or smooth muscle cell function (atherosclerosis and obesity); a vascular disease [atheromatous ateriosclerosis, nonatheromatous ateriosclerosis, ischemic heart disease including myocardial infarction and peripheral arterial occlusion, Raynaud's disease and phenomenon, and thromboangiitis obliterans (Buerger's disease)]; chronic arthritis; inflammatory bowel diseases; skin dermatoses; diabetes mellitus; SSAO-mediated complication [diabetes (insulin dependent diabetes mellitus (IDDM) and non-insulin dependent diabetes mellitus (NIDDM)) and vascular complication (heart attack, angina, strokes, amputations, blindness and renal failure)]; macular edema (e.g., diabetic and non-diabetic macular edema); hepatitis; transplantation; and the like.

The “preventing or treating a vascular adhesion protein-1 (VAP-1) associated disease” and “prophylaxis or treatment of a vascular adhesion protein-1 (VAP-1) associated disease”, particularly “preventing or treating macular edema” and “prophylaxis or treatment of macular edema”, are intended to include administration of a compound having VAP-1 inhibitory activity (i.e. VAP-1 inhibitor) to a subject for therapeutic purposes, which may include prophylaxis, amelioration, prevention and cure of the above described VAP-1 associated diseases, particularly macular edema. As used herein, by the “subject” is meant a target of the administration of a VAP-1 inhibitor in the present invention, which is specifically various animals such as mammal, e.g., human, mouse, rat, swine, dog, cat, horse, bovine and the like, especially human.

The above methods comprise administration of a VAP-1 inhibitor in an amount sufficient to treat the VAP-1 associated disease, especially macular edema. Any VAP-1 inhibitor can be used in the method of the present invention as long as it is safe and effective. Herein, the “VAP-1 inhibitor” will be used to refer to such compounds/medicaments, which include Compound (I), and is intended to encompass all compounds that inhibit enzyme activity of VAP-1 at any and all points in the action mechanism thereof.

For example, the VAP-1 inhibitor used in the resent invention may further include fluoroallylamine derivatives, semicarbazide derivatives, hydrazide derivatives, hydrazino derivatives, 1,3,4-oxadiazine derivatives, 4-alkyl-5-alkoxycarbonyl-4,5,6,7-tetrahydroimidazo[4,5-c]pyridine derivatives, 2,6-diethoxybenzylamine, 2,6-di(n-propoxy)benzylamine, 2,6-diisopropoxybenzylamine, 2,6-di(n-butoxy)benzylamine, 2,6-bis(methoxymethoxy)benzylamine, 2,6-bis(methoxymethyl)benzylamine, 2,6-diethylbenzylamine, 2,6-di-n-propylbenzylamine, 2,6-bis(2-hydroxyethoxy)benzylamine, and the like.

The above compounds are exemplified by the following.

1) fluoroallylamine derivatives, semicarbazide derivatives and hydrazide derivatives described in WO 93/23023,

2) hydrazino derivatives described in WO 02/02090,

3) 1,3,4-oxadiazine derivatives described in WO 02/02541,

4) 4-alkyl-5-alkoxycarbonyl-4,5,6,7-tetrahydroimidazo[4,5-c]pyridine derivatives described in WO 02/38153,

5) 2,6-diethoxybenzylamine, 2,6-di(n-propoxy)benzylamine, 2,6-diisopropoxybenzylamine, 2,6-di(n-butoxy)benzylamine, 2,6-bis(methoxymethoxy)benzylamine, 2,6-bis(methoxymethyl)benzylamine, 2,6-diethylbenzylamine, 2,6-di-n-propylbenzylamine and 2,6-bis(2-hydroxyethoxy)benzylamine described in U.S. Pat. No. 4,888,283.

The compounds exemplified in the description of the present invention, in WO 93/23023 as an SSAO inhibitor, such as those described by Lyles et al. (Biochem. Pharmacol. 36:2847, 1987), and in U.S. Pat. No. 4,650,907, U.S. Pat. No. 4,916,151, U.S. Pat. No. 4,943,593, U.S. Pat. No. 4,965,288, U.S. Pat. No. 5,021,456, U.S. Pat. No. 5,059,714, U.S. Pat. No. 4,699,928, European patent application No. 0295604, European patent application No. 0224924 and European patent application No. 0168013, are also encompassed in the VAP-1 inhibitor.

Of the above-mentioned compounds, preferred are Compound (I) and derivatives thereof, and more preferred are
N-(4-{2-[5-({[amino(imino)methyl]amino}methyl)-2-thienyl]ethyl}thiazol-2-yl)acetamide hydrochloride,
2-{5-[2-(2-acetylaminothiazol-4-yl)ethyl]-2-thienyl}-N-[amino(imino)methyl]acetamide,
N-[4-(2-{4-[(2-amino-1H-imidazol-4-yl)methyl]phenyl}ethyl)thiazol-2-yl]acetamide, and derivatives thereof.

The term “derivative” is intended to include all compounds derived from the original compound.

In the present invention, the VAP-1 inhibitor can be administered as a prodrug to a subject. The term “prodrug” is intended to include all compounds that convert to the VAP-1 inhibitor in the body of the administration subject. The prodrug can be any pharmaceutically acceptable prodrug of VAP-1 inhibitor.

Moreover, the VAP-1 inhibitor can be administered to the administration subject as a pharmaceutically acceptable salt.

The pharmaceutically acceptable salt of the VAP-1 inhibitor is nontoxic and a pharmaceutically acceptable conventional salt, which is exemplified by salts with inorganic or organic base, such as alkali metal salt (e.g., sodium salt, potassium salt and the like), alkaline earth metal salt (e.g., calcium salt, magnesium salt and the like), ammonium salt, and amine salt (e.g., triethylamine salt, N-benzyl-N-methylamine salt and the like).

In addition, the pharmaceutically acceptable salt of the VAP-1 inhibitor includes a pharmaceutically acceptable acid addition salt. Examples of the pharmaceutically acceptable acid addition salts include those derived from mineral acids, such as hydrochloric, hydrobromic, hydriodic, phosphoric, metaphosphoric, nitric and sulfuric acids, and organic acids, such as tartaric, acetic, citric, malic, lactic, fumaric, benzoic, glycolic, gluconic, succinic and arylsulfonic acids such as p-toluenesulfonic acid.

As a pharmaceutically acceptable salt of the VAP-1 inhibitor represented by the formula (I), a pharmaceutically acceptable acid addition salt such as hydrochloride and hydriodide, particularly (mono-, di- or tri-)hydrochloride, is preferable.

Some VAP-1 inhibitors except Compound (I) may be commercially available or can be produced based on known references.

Compound (I) can be prepared according to Production Method given below, Reference Example, Production Examples, analogous methods thereto and the organic synthetic methods known to the art.

The VAP-1 inhibitor or a pharmaceutically acceptable salt thereof can be administered in accordance with the present inventive method via any suitable route. Suitable routes of administration include systemic, such as oral or by injection, topical, periocular (e.g., subTenon's), subconjunctival, intraocular, subretinal, suprachoroidal and retrobulbar administrations. The manner in which the VAP-1 inhibitor is administered is dependent, in part, upon whether the treatment of a VAP-1 associated disease is prophylactic or therapeutic.

The VAP-1 inhibitor is preferably administered as soon as possible after it has been determined that a subject such as mammal, specifically a human, is at risk for a VAP-1 associated disease (prophylactic treatments) or has begun to develop a VAP-1 associated disease (therapeutic treatments). Treatment will depend, in part, upon the particular VAP-1 inhibitor to be used, the amount of the VAP-1 inhibitor to be administered, the route of administration, and the cause and extent, if any, of a VAP-1 associated disease realized.

One of ordinary skill in the art will appreciate that suitable methods of administering a VAP-1 inhibitor, which is useful in the present inventive method, are available. Although more than one route can be used to administer a particular VAP-1 inhibitor, a particular route can provide a more immediate and more effective reaction than a different route. Accordingly, the described routes of administration are merely exemplary and are in no way limiting.

The dose of the VAP-1 inhibitor administered to the administration subject such as animal including human, particularly a human, in accordance with the present invention, should be sufficient to effect the desired response in the subject over a reasonable time frame. One of ordinary skill in the art will recognize that dosage will depend upon a variety of factors, including the strength of the particular VAP-1 inhibitor to be employed; the age, species, conditions or disease states, and body weight of the subject; and the degree of a VAP-1 associated disease. The size of the dose also will be determined depending on the route, timing and frequency of administration; the existence, nature and extent of any adverse side effects that might accompany the administration of a particular VAP-1 inhibitor; and the desired physiological effect. It will be appreciated by one of ordinary skill in the art that various conditions or disease states may require prolonged treatment involving multiple administrations.

Suitable doses and dosage regimens can be determined by conventional range-finding techniques known to one of ordinary skill in the art. Generally, treatment is initiated with smaller dosages, which are less than the optimum dose of the compound. Thereafter, the dosage is increased by small increments until the optimum effect under the circumstances is reached.

Generally, the VAP-1 inhibitor can be administered in the dose of from about 1 μg/kg/day to about 300 mg/kg/day, preferably from about 0.1 mg/kg/day to about 10 mg/kg/day, which is given in a single dose or 2 to 4 doses a day or in a sustained manner.

Pharmaceutical compositions for use in the present inventive method preferably comprise a “pharmaceutically acceptable carrier” and an amount of a VAP-1 inhibitor sufficient to treat a VAP-1 associated disease, especially macular edema, prophylactically or therapeutically as an active ingredient. The carrier can be any of those conventionally used and is limited only by chemico-physical considerations, such as solubility and lack of reactivity of the compound, and by the route of administration.

The VAP-1 inhibitor can be administered in various manners to achieve the desired VAP-1 inhibitory effect. The VAP-1 inhibitor can be administered alone or in combination with pharmaceutically acceptable carriers or diluents, the properties and nature of which are determined by the solubility and chemical properties of the inhibitor selected, the chosen administration route, and standard pharmaceutical practice. The VAP-1 inhibitor may be administered orally in solid dosage forms, e.g., capsules, tablets, powders, or in liquid forms, e.g., solutions or suspensions. The inhibitor may also be injected parenterally in the form of sterile solutions or suspensions. Solid oral forms may contain conventional excipients, for instance, lactose, sucrose, magnesium stearate, resins, and like materials. Liquid oral forms may contain various flavoring, coloring, preserving, stabilizing, solubilizing or suspending agents. Parenteral preparations are sterile aqueous or non-aqueous solutions, or suspensions which may contain certain various preserving, stabilizing, buffering, solubilizing or suspending agents. If desired, additives such as saline or glucose may be added to make the solutions isotonic.

The present inventive method also can involve the co-administration of other pharmaceutically active compound(s). By “co-administration” is meant administration of the other pharmaceutically active compound(s) before, concurrently with, e.g., in combination with a VAP-1 inhibitor in the same formulation or in separate formulations, or after administration of the VAP-1 inhibitor as described above. For example, corticosteroids, prednisone, methylprednisolone, dexamethasone or triamcinolone acetinide, or noncorticosteroid anti-inflammatory compounds, such as ibuprofen or flubiprofen, can be co-administered. Similarly, vitamins and minerals (e.g., zinc), anti-oxidants (e.g., carotenoids (such as a xanthophyll carotenoid like zeaxanthin or lutein)), and micronutrients can be co-administered.

In addition, the VAP-1 inhibitor according to the present invention is useful for preparing a medicament such as a therapeutic or prophylactic agent for the VAP-1 associated diseases.

Compound (I) can be synthesized according to the Production Method given below.

Production Method

Compound (I) is prepared in accordance with, but is not limited to, the following procedures. Those skilled in the art will recognize that the procedures can be modified according to the conventional methods known per se.
Procedure A: Synthesis of Compound (1) wherein Y is a bond and is —CONH— 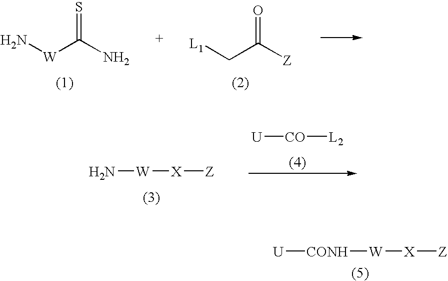
wherein
L₁ is a leaving group such as halogen;
U, W and Z are as defined above and Z may be acyloxy(lower alkyl) [e.g., acetoxymethyl];
X is as defined above, in this case, 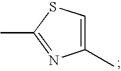
and
L₂ is a leaving group such as —OH, halogen, —O-acyl (e.g., —O-acetyl and the like).
Formation of Thiazole Moiety X

Compound (1) is reacted with Compound (2) or its salt to give Compound (3).

Suitable salt of Compound (2) may be the same as those exemplified for Compound (I).

Compounds (1) and (2) or salt thereof may be commercially available or can be prepared in accordance with the methods known per se (see, e.g., Reference Example).

The reaction is usually carried out in a conventional solvent such as ethanol, acetone, dichloromethane, acetic acid, and other organic solvent which does not adversely affect the reaction, or a mixture thereof.

The reaction temperature is not critical, and the reaction can be carried out under cooling to heating.

Compound (3) thus obtained can be isolated or purified by known separation or purification means, such as concentration, concentration in vacuo, solvent extraction, crystallization, recrystallization, phase transfer, chromatography and the like, and can be converted to a salt same as those exemplified for Compound (I).

Acylation

Compound (3) or its salt is reacted with Compound (4) to give Compound (5). This reaction is an acylation.

The conventional acylation method may be employed in the present invention.

Compound (4) may be commercially available or can be prepared in accordance with the methods known per se.

The reaction is usually carried out in a conventional solvent such as dichloromethane, chloroform and methanol, and other organic solvent which does not adversely affect the reaction, or a mixture thereof.

The reaction is also preferably carried out in the presence of a conventional base such as 4-dimethylaminopyridine, pyridine, etc. A liquid base can be also used as the solvent.

The reaction temperature is not critical, and the reaction can be carried out under cooling to heating.

Compound (5) thus obtained can be isolated or purified by known separation or purification means, such as concentration, concentration in vacuo, solvent extraction, crystallization, recrystallization, phase transfer, chromatography and the like, and can be converted to a salt same as those exemplified for Compound (I).

The acylation may be applied to Compound (1) in advance.

The nitrogen atom(s) in Compound (1), (2), (3) or (5) may be protected or deprotected, as necessary, in accordance with the methods known per se such as the methods described in Protective Groups in Organic Synthesis, published by John Wiley and Sons (1980), and the like.
Procedure B: Synthesis of Compound (1) wherein Y is lower alkylene such as ethylene 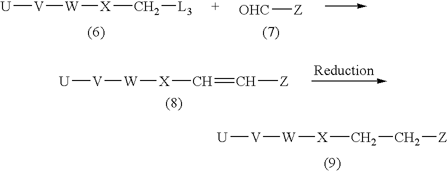
wherein
L₃ is a leaving group such as halogen and/or halogenotriphenylphosphinyl (e.g., ClPh₃P—, BrPh₃P— and the like);
U, V, W and X are as defined above;
Z are as defined above provided that R² in Z may be —CHO.
Formation of Olefin Compound

Compound (6) or its salt is reacted with Compound (7) or its salt to give an olefin compound (8).

Suitable salts of Compounds (6) and (7) may be the same as those exemplified for Compound (I).

Compounds (6) and (7) or salts thereof may be commercially available or can be prepared in accordance with the methods known per se (see, e.g., Production Example 1).

The reaction is usually carried out in a conventional solvent such as N,N-dimethylformamide, dimethylsulfoxide, tetrahydrofuran and dichloromethane, and other organic solvent which does not adversely affect the reaction, or a mixture thereof.

The reaction is also usually carried out in the presence of triphenylphosphine and a conventional base such as potassium tert-butoxide, sodium hydride, sodium hydroxide and the like.

The reaction temperature is not critical, and the reaction can be carried out under cooling to heating.

Compound (8) thus obtained can be isolated or purified by known separation or purification means, such as concentration, concentration in vacuo, solvent extraction, crystallization, recrystallization, phase transfer, chromatography and the like, and can be converted to a salt same as those exemplified for Compound (I).

Reduction

Compound (8) or its salt is reduced in accordance with a conventional method to give Compound (9).

The conventional reduction includes hydrogenation, catalytic hydrogenation, etc.

Among others, catalytic hydrogenation is preferable.

The catalytic hydrogenation is carried out in the presence of a catalyst such as palladium on carbon, preferably 10% palladium on carbon.

The catalytic hydrogenation is usually carried out in a conventional solvent such as tetrahydrofuran, ethanol, ethyl acetate, and other solvent which does not adversely affect the reaction, or a mixture thereof.

The catalytic hydrogenation is also preferably carried out in the presence of a conventional acid such as acetic acid, hydrochloric acid and the like. A liquid acid can be also used as the solvent.

The reaction temperature is not critical, and the reaction can be carried out under cooling to heating.

Compound (9) thus obtained can be isolated or purified by known separation or purification means, such as concentration, concentration in vacuo, solvent extraction, crystallization, recrystallization, phase transfer, chromatography and the like, and can be converted to a salt same as those exemplified for Compound (I).

Therefore, as indicated in the following scheme, Compound (11) or a salt thereof can be prepared from Compound (10) or a salt thereof in a similar manner as described above. Suitable salts of Compounds (10) and (11) may be the same as those exemplified for Compound (I).

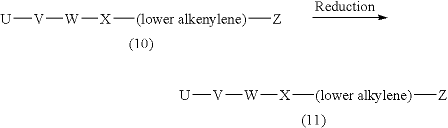

The nitrogen atom(s) in Compound (6), (7), (8), (9), (10) or (11) may be protected or deprotected, as necessary, in accordance with the methods known per se such as the methods described in Protective Groups in Organic Synthesis, published by John Wiley and Sons (1980), and the like.

The present invention is explained in more detail in the following by way of Reference Example, Production Examples and Example, which are not to be construed as limitative.

Test Compounds used in Example were N-(4-{2-[5-({[amino(imino)methyl]amino}methyl)-2-thienyl]ethyl}thiazol-2-yl)acetamide hydrochloride, 2-{5-[2-(2-acetylaminothiazol-4-yl)ethyl]-2-thienyl}-N-[amino(imino)methyl]acetamide and N-[4-(2-{4-[(2-amino-1H-imidazol-4-yl)methyl]phenyl}ethyl)thiazol-2-yl]acetamide, which were prepared in Production Examples 3, 4 and 17, respectively.

### REFERENCE EXAMPLE

### Synthesis of N-{4-[2-(4-{[amino(imino)methyl]amino}phenyl)ethyl]thiazol-2-yl}acetamide

Step 1

A mixture of 3-chloro-2-oxopropyl acetate (5 g) and thiourea (2.5 g) in ethanol (25 ml) was refluxed for 4 hours. The reaction mixture was cooled to ambient temperature and the resulting crystalline precipitate was collected by filtration and washed with ethanol (20 ml) to give (2-aminothiazol-4-yl)methyl acetate hydrochloride (3.5 g) as white crystals.

¹H-NMR (DMSO-d₆), δ (ppm): 2.07 (3H, s), 4.92 (2H, s), 6.87 (1H, s).

MS: 173 (M+H)⁺

Step 2

To a mixture of (2-aminothiazol-4-yl)methyl acetate hydrochloride (56 g) and pyridine (45 g) in dichloromethane (560 ml) was added acetyl chloride (23 g) over a period of 30 minutes at 5° C., and the reaction mixture was stirred at the same temperature for 10 minutes. The reaction mixture was poured into water (500 ml) and extracted with chloroform (1 L). The organic layer was dried over sodium sulfate and concentrated in vacuo. The residual solid was collected by filtration and washed with isopropyl ether to give (2-acetylaminothiazol-4-yl)methyl acetate (47 g) as white crystals.

¹H-NMR (CDCl₃), δ (ppm): 2.12 (3H, s), 2.29 (3H, s), 5.08 (2H, s), 6.93 (1H, s).

MS: 215 (M+H)⁺

Step 3

A mixture of (2-acetylaminothiazol-4-yl)methyl acetate (46 g) and potassium carbonate (30 g) in methanol (640 ml) was stirred at ambient temperature for 3 hours. The reaction mixture was concentrated in vacuo. The residue was diluted with chloroform, and the insoluble material was filtered off. The resulting solution was purified by flash column chromatography over silica-gel with methanol/chloroform (1:99). The resulted solid was collected by filtration and washed with isopropyl ether to give N-(4-hydroxymethylthiazol-2-yl)acetamide (35 g) as white crystals.

¹H-NMR (DMSO-d₆), δ (ppm): 2.12 (3H, s), 4.44 (2H, d, J=5.0 Hz), 5.20 (1H, t, J=5.0 Hz), 6.88 (1H, s), 12.02 (1H, brs).

MS: 173 (M+H)⁺

Step 4

N-(4-Hydroxymethylthiazol-2-yl)acetamide (2.8 g) was dissolved in methanol (10 ml) and chloroform (200 ml). Then manganese (IV) oxide (28.3 g) was added to the solution under nitrogen atmosphere. The reaction mixture was stirred at room temperature for 7 hours and filtered through a Celite pad. The filtrate was concentrated in vacuo. The resulting solid was washed with ethyl ether to give N-(4-formylthiazol-2-yl)acetamide (2.01 g) as an off-white solid.

mp. 195.5-199° C.

¹H-NMR (DMSO-d₆), δ (ppm): 2.17 (3H, s), 8.28 (1H, s), 9.79 (1H, s), 12.47 (1H, brs).

Step 5

1-(Bromomethyl)-4-nitrobenzene (1.9 g), triphenylphosphine (2.31 g) and N,N-dimethylformamide (20 ml) were combined under nitrogen atmosphere. The reaction mixture was stirred at room temperature for 2.5 hours. Then potassium tert-butoxide (1.19 g) and N-(4-formylthiazol-2-yl)acetamide (1.5 g) obtained in Step 4 were added and the mixture was stirred at room temperature for 14 hours. The reaction mixture was poured into ice-water and extracted with ethyl acetate. The organic layer was washed with 1N hydrochloric acid, water and saturated sodium chloride solution, dried over anhydrous magnesium sulfate, and concentrated in vacuo. The residue was purified by flash column chromatography over silica gel with n-hexane/ethyl acetate (1:1→1:2) as an eluent, and triturated with ethyl ether to give N-{4-[(Z)-2-(4-nitrophenyl)ethenyl]thiazol-2-yl}acetamide (1.59 g) as a yellow solid.

mp. 155-157° C.

¹H-NMR (DMSO-d₆), δ (ppm): 2.13 (3H, s), 6.64 (1H, d, J=12.5 Hz), 6.71 (1H, d, J=12.5 Hz), 7.18 (1H, s), 7.79 (2H, d, J=9.0 Hz), 8.17 (2H, d, J=9.0 Hz), 12.02 (1H, brs).

MS: 290 (M+H)⁺

Step 6

A mixture of N-{4-[(Z)-2-(4-nitrophenyl)ethenyl]thiazol-2-yl}acetamide (2 g) and 10% palladium on carbon (400 mg) in methanol (25 ml), tetrahydrofuran (25 ml) and acetic acid (18 ml) was stirred under 4 atm hydrogen at ambient temperature or 5 hours. The reaction mixture was filtered through a elite pad, and the filtrate was concentrated in vacuo. The residue was dissolved in ethyl acetate. The organic solution as washed with saturated sodium hydrogen carbonate solution and saturated sodium chloride solution, dried over anhydrous magnesium sulfate, and concentrated in vacuo. The residue was purified by flash column chromatography over silica gel with n-hexane/ethyl acetate (1:2)→ethyl acetate as an eluent, and triturated with ethyl alcohol/ethyl ether to give N-{4-[2-(4-aminophenyl)ethyl]thiazol-2-yl}acetamide (539.6 mg) as an off-white solid.

mp. 102.5-104° C.

¹H-NMR (DMSO-d₆), δ (ppm): 2.11 (3H, s), 2.75 (4H, brs), 4.82 (2H, s), 6.46 (2H, d, J=8.5 Hz), 6.69 (1H, s), 6.83 (2H, d, J=8.5 Hz), 12.07 (1H, brs).

MS: 262 (M+H)⁺

Step 7

To a suspension of N-{4-[2-(4-aminophenyl)ethyl]thiazol-2-yl}acetamide (26 g) in ethanol (500 ml) were added 4N hydrogen chloride in ethyl acetate (25 ml) and cyanamide (6.3 g). The mixture was refluxed for 26 hours. The reaction mixture was cooled to ambient temperature and poured into a mixture of ethyl acetate (500 ml) and saturated sodium hydrogen carbonate solution (500 ml). The resulting precipitate was collected by filtration and washed with water (300 ml) and ethanol (300 ml) to give N-{4-[2-(4-{[amino(imino)methyl]amino}phenyl)ethyl]thiazol-2-yl}acetamide (18 g) as white crystals.

¹H-NMR (DMSO-d₆), δ (ppm): 2.10 (3H, s), 2.85 (4H, s), 6.79 (1H, s), 6.83 (2H, d, J=7 Hz), 7.10 (2H, d, J=7 Hz).

MS: 304 (M+H)⁺

### Production Example 1

### Synthesis of N-(4-{2-[5-(2-{[amino(imino)methyl]amino}ethyl)-2-thienyl]ethyl}thiazol-2-yl)acetamide

Step 1

2,5-Thiophenedicarbaldehyde (2.14 g), methyl (triphenylphosphoranylidene)acetate (5.11 g) and trichloromethane (20 ml) were combined at room temperature under nitrogen atmosphere, and the reaction mixture was refluxed for 1 hour. The solvent was removed in vacuo. The residue was purified by flash column chromatography over silica gel with n-hexane/ethyl acetate (1:1) as an eluent to give methyl (2E)-3-(5-formyl-2-thienyl)acrylate (2.5 g) as an off-white solid.

mp. 61-62.5° C.

¹H-NMR (DMSO-d₆), δ (ppm): 3.74 (3H, s), 6.60 (1H, d, J=16.0 Hz), 7.75 (1H, d, J=4.0 Hz), 7.87 (1H, d, J=16.0 Hz), 8.02 (1H, d, J=4.0 Hz), 9.94 (1H, s)

MS: 197 (M+H)⁺

Step 2

[(2-Acetylaminothiazol-4-yl)methyl](triphenyl)phosphonium chloride (6.72 g) and dimethylformamide (50 ml) were combined under nitrogen atmosphere, and potassium tert-butoxide (1.79 g) was then added to the suspension at 0° C. The reaction mixture was stirred at 0° C. for 15 minutes, and methyl (2E)-3-(5-formyl-2-thienyl)acrylate (2.24 g) was added to the mixture at 0° C. The reaction mixture was stirred at room temperature for 2.5 hours. Water was added to the mixture, and the precipitate was collected in vacuo to give methyl (2E)-3-{5-[(E)-2-(2-acetylaminothiazol-4-yl)vinyl]-2-thienyl}acrylate (4.55 g) as a yellow solid.

mp. 200-202° C.

¹H-NMR (DMSO-d₆), δ (ppm): 2.16 (3H, s), 3.72 (3H, s), 6.21 (1H, d, J=15.5 Hz), 6.99 (1H, d, J=15.5 Hz), 7.25 (1H, d, J=4.0 Hz), 7.27 (1H, s), 7.34 (1H, d, J=15.5 Hz), 7.51 (1H, d, J=4.0 Hz), 7.79 (1H, d, J=15.5 Hz), 12.22 (1H, s).

MS: 335 (M+H)

Step 3

Methyl (2E)-3-{5-[(E)-2-(2-acetylaminothiazol-4-yl)vinyl]-2-thienyl}acrylate (4.5 g), 10% palladium on carbon (4.71 g), methanol (10 ml) and dimethylformamide (45 ml) were combined under nitrogen atmosphere. The reaction mixture was stirred at room temperature for 10 hours under hydrogen atmosphere (4 atm) and filtered through a Celite pad. The filtrate was concentrated in vacuo. The residue was purified by flash column chromatography over silica gel with trichloromethane/methanol (20:1→10:1) as an eluent to give methyl 3-{5-[2-(2-acetylaminothiazol-4-yl)ethyl]-2-thienyl}propanoate (2.09 g) as a pale yellow solid.

mp. 104.5-106° C.

¹H-NMR (DMSO-d₆), δ (ppm): 2.11 (3H, s), 2.65 (2H, t, J=7.0 Hz), 2.81-3.12 (6H, m), 3.59 (3H, s), 6.63 (2H, s), 6.77 (1H, s), 12.08 (1H, s).

MS: 339 (M+H)

Step 4

Methyl 3-{5-[2-(2-acetylaminothiazol-4-yl)ethyl]-2-thienyl}propanoate (2 g), 1N sodium hydroxide solution (14.8 ml) and 1,4-dioxane (20 ml) were combined at 0° C., and the reaction mixture was stirred at room temperature for 1 hour. The organic solvent was evaporated in vacuo. The residual aqueous solution was acidified with 1N hydrochloric acid. The precipitate was collected in vacuo. The solid was washed with ethyl ether to give 3-{5-[2-(2-acetylaminothiazol-4-yl)ethyl]-2-thienyl}propanoic acid (1.64 g) as a pale yellow solid.

mp. 163.5-165° C.

¹H-NMR (DMSO-d₆), δ (ppm): 2.11 (3H, s), 2.46-2.57 (2H, m), 2.82-2.99 (4H, m), 3.00-3.12 (2H, m), 6.63 (2H, s), 6.77 (1H, s), 12.08 (1H, brs), 12.18 (1H, brs).

MS: 325 (M+H)

Step 5

3-{5-[2-(2-Acetylaminothiazol-4-yl)ethyl]-2-thienyl}propanoic acid (700 mg), triethylamine (0.451 ml) and t-butyl alcohol (10 ml) were combined under nitrogen atmosphere. Diphenylphosphoryl azide (0.558 ml) was added dropwise to the solution at room temperature. The reaction mixture was refluxed for 4 hours and cooled to room temperature. The mixture was diluted with ethyl acetate. The organic solution was washed with 1N hydrochloric acid, water and brine, dried over anhydrous magnesium sulfate, and concentrated in vacuo. The residue was purified by flash column chromatography over silica gel with n-hexane/ethyl acetate (1:1) as an eluent to give tert-butyl (2-{5-[2-(2-acetylaminothiazol-4-yl)ethyl]-2-thienyl}ethyl)carbamate (211.3 mg) as a pale yellow wax.

¹H-NMR (DMSO-d₆), δ (ppm): 1.37 (9H, s), 2.11 (3H, s), 2.72-3.18 (8H, m), 6.60-6.66 (2H, m), 6.78 (1H, s), 6.93 (1H, t, J=5.5 Hz), 12.08 (1H, s).

MS: 396 (M+H)

Step 6

tert-Butyl (2-{5-[2-(2-acetylaminothiazol-4-yl)ethyl]-2-thienyl}ethyl)carbamate (201.4 mg), 4N hydrochloric acid in 1,4-dioxane solution (2 ml) and methanol (1 ml) were combined under nitrogen atmosphere. The reaction mixture was stirred at room temperature for 2 hours and concentrated in vacuo. The residue, di-tert-butyl (1H-pyrazol-1-ylmethylidene)biscarbamate (158 mg), N,N-diisopropylethylamine (0.177 ml), tetrahydrofuran (3 ml) and dimethylformamide (1 ml) were combined under nitrogen atmosphere. The reaction mixture was stirred at room temperature for 2 hours and concentrated in vacuo. The residue was purified by flash column chromatography over silica gel with n-hexane/ethyl acetate (1:1) as an eluent to give di-tert-butyl {[(2-{5-[2-(2-acetylaminothiazol-4-yl)ethyl]-2-thienyl}ethyl)amino]methylidene}biscarbamate (147.2 mg) as pale yellow amorphous.

¹H-NMR (DMSO-d₆), δ (ppm): 1.40 (9H, s), 1.47 (9H, s), 2.11 (3H, s), 2.88 (2H, t, J=7.0 Hz), 2.94 (2H, t, J=7.0 Hz), 3.09 (2H, t, J=7.0 Hz), 3.50 (2H, dt, J=5.5, 7.0 Hz), 6.65 (1H, d, J=3.5 Hz), 6.67 (1H, d, J=3.5 Hz), 6.76 (1H, s), 8.40 (1H, t, J=5.5 Hz), 11.50 (1H, s), 12.08 (1H, s).

MS: 538 (M+H)

Step 7

Di-tert-butyl {[(2-{5-[2-(2-acetylaminothiazol-4-yl)ethyl]-2-thienyl}ethyl)amino]methylidene}biscarbamate (137.2 mg), 4N hydrochloric acid solution (3 ml) in 1,4-dioxane, and methanol (1 ml) were combined under nitrogen atmosphere. The reaction mixture was stirred at room temperature for 7 hours. The solvent was removed in vacuo. The residue was dissolved in water and ethyl acetate. The mixture was made basic (pH=8) with saturated aqueous sodium hydrogen carbonate solution. The precipitate was collected in vacuo. The solid was washed with acetonitrile to give N-(4-{2-[5-(2-{[amino(imino)methyl]amino}ethyl)-2-thienyl]ethyl}thiazol-2-yl)acetamide (29.3 mg) as an off-white solid.

mp. 121.5-123° C.

¹H-NMR (DMSO-d₆), δ (ppm): 2.02 (3H, s), 2.86 (2H, t, J=7.0 Hz), 2.94 (2H, m), 3.07 (2H, t, J=7.0 Hz), 3.20-3.60 (2H, m), 6.59 (1H, brs), 6.66 (1H, s), 6.70 (1H, brs).

MS: 338 (M+H)

### Production Example 2

### Synthesis of N-(4-{2-[5-(3-{[amino(imino)methyl]amino}propyl)-2-thienyl]ethyl}thiazol-2-yl)acetamide hydrochloride

Step 1

To a stirred solution of 3-{5-[2-(2-acetylaminothiazol-4-yl)ethyl]-2-thienyl}propanoic acid (500 mg), obtained in Step 4 of Production Example 1, in dry tetrahydrofuran (5 ml) was added dropwise 2M borane-methyl sulfide complex solution in tetrahydrofuran (2.3 ml) at 0° C. under nitrogen atmosphere. The reaction mixture was stirred at room temperature for 3 hours and the reaction was then quenched with methanol. 1N Hydrochloric acid was added to the mixture, and the mixture was stirred at 70° C. for 1 hour. The mixture was extracted twice with ethyl acetate. The combined organic layer was washed with saturated aqueous sodium hydrogen carbonate solution and brine, dried over anhydrous magnesium sulfate, and concentrated in vacuo. The residue was purified by flash column chromatography over silica gel with trichloromethane/methanol (20:1) as an eluent to give N-(4-{2-[5-(3-hydroxypropyl)-2-thienyl]ethyl}thiazol-2-yl)acetamide (465.8 mg) as a pale yellow wax.

¹H-NMR (DMSO-d₆), δ (ppm): 1.61-1.83 (2H, m), 2.12 (3H, s), 2.73 (2H, t, J=7.5 Hz), 2.79-2.95 (2H, m), 3.00-3.13 (2H, m), 3.42 (2H, m), 4.48 (1H, t, J=5.0 Hz), 6.60 (1H, d, J=3.5 Hz), 6.62 (1H, d, J=3.5 Hz), 6.78 (1H, s), 12.07 (1H, s).

MS: 311 (M+H)

Step 2

N-(4-{2-[5-(3-Hydroxypropyl)-2-thienyl]ethyl}thiazol-2-yl)acetamide (260.8 mg) obtained in Step 1 of this Production Example, carbon tetrabromide (417.9 mg), triphenylphosphine (330.5 mg) and tetrahydrofuran (3 ml) were combined at 0° C. under nitrogen atmosphere. The reaction mixture was stirred at room temperature for 1 hour, and the precipitate was filtered off. The filtrate was concentrated in vacuo. The residue was purified by flash column chromatography over silica gel with n-hexane/ethyl acetate (1:2) as an eluent. The residual solid, potassium phthalimide (155.6 mg) and dimethylformamide (3 ml) were combined under nitrogen atmosphere. The reaction mixture was stirred at 50° C. for 3 hours. After cooling to room temperature, ethyl acetate and 1N hydrochloric acid were added to the reaction mixture. The organic layer was washed with water, saturated sodium hydrogen carbonate and brine, dried over anhydrous magnesium sulfate, and concentrated in vacuo. The residue was purified by flash column chromatography over silica gel with trichloromethane/methanol (20:1) as an eluent to give N-[4-(2-{5-[3-(1,3-dioxo-1,3-dihydro-2H-isoindol-2-yl)propyl]-2-thienyl}ethyl)thiazol-2-yl]acetamide (177.6 mg) as pale yellow amorphous.

¹H-NMR (DMSO-d₆), δ (ppm): 1.90 (2H, m), 2.11 (3H, s), 2.71 (2H, t, J=7.0 Hz), 2.79-2.92 (2H, m), 2.892-3.10 (2H, m), 3.62 (2H, t, J=7.0 Hz), 6.59 (1H, d, J=3.5 Hz), 6.64 (1H, d, J=3.5 Hz), 6.77 (1H, s), 7.78-7.90 (4H, m), 12.07 (1H, s).

MS: 440 (M+H)

Step 3

N-[4-(2-{5-[3-(1,3-Dioxo-1,3-dihydro-2H-isoindol-2-yl)propyl]-2-thienyl}ethyl)thiazol-2-yl]acetamide (158 mg), hydrazine monohydrate (0.174 ml) and acetonitrile (2 ml) were combined under nitrogen atmosphere. The reaction mixture was stirred at 50° C. for 1 hour. After cooling to room temperature, the mixture was diluted with trichloromethane. The precipitate was filtered off. The filtrate was concentrated in vacuo. The residue was purified by flash column chromatography over NH silica gel with trichloromethane/methanol (20:1) as an eluent to give N-(4-{2-[5-(3-aminopropyl)-2-thienyl]ethyl}thiazol-2-yl)acetamide (108 mg) as a pale yellow solid.

mp. 106.5-108° C.

¹H-NMR (DMSO-d₆), δ (ppm): 1.61 (2H, m), 2.11 (3H, s), 2.54 (2H, t, J=7.0 Hz), 2.72 (2H, t, J=7.0 Hz), 2.82-2.94 (2H, m), 3.00-3.13 (2H, m), 6.59 (1H, d, J=3.5 Hz), 6.62 (1H, d, J=3.5 Hz), 6.77 (1H, s).

MS: 310 (M+H)

Step 4

N-(4-{2-[5-(3-Aminopropyl)-2-thienyl]ethyl}thiazol-2-yl)acetamide (102.8 mg), N,N′-bis(tert-butoxycarbonyl)-1H-pyrazole-1-carboxamidine (103.1 mg) and tetrahydrofuran (2 ml) were combined under nitrogen atmosphere. The reaction mixture was stirred at room temperature for 4 hours and concentrated in vacuo. The residue was purified by preparative silica gel chromatography with trichloromethane/methanol (30:1) as an eluent to give di-tert-butyl {[(3-{5-[2-(2-acetylaminothiazol-4-yl)ethyl]-2-thienyl}propyl)amino]methylidene}biscarbamate (160.1 mg) as colorless amorphous.

¹H-NMR (DMSO-d₆), δ (ppm): 1.39 (9H, s), 1.48 (9H, s), 1.7-1.9 (2H, m), 2.11 (3H, s), 2.72 (2H, t, J=7 Hz), 2.82-2.94 (2H, m), 2.82-3.01 (2H, m), 3.25-3.38 (2H, m), 6.62 (1H, d, J=4 Hz), 6.67 (1H, d, J=4 Hz), 6.77 (1H, s), 8.32 (1H, s), 11.48 (1H, s), 12.07 (1H, s).

MS: 552 (M+H)

Step 5

Di-tert-butyl {[(3-{5-[2-(2-acetylaminothiazol-4-yl)ethyl]-2-thienyl}propyl)amino]methylidene}biscarbamate (144.7 mg), methanol (1 ml) and 4N hydrochloric acid solution (3 ml) in 1,4-dioxane were combined under nitrogen atmosphere. The reaction mixture was stirred at room temperature for 17 hours. The solvent was removed in vacuo. The residue was washed with ethyl acetate to give N-(4-{2-[5-(3-{[amino(imino)methyl]amino}propyl)-2-thienyl]ethyl}thiazol-2-yl)acetamide hydrochloride (76.8 mg) as off-white amorphous.

¹H-NMR (DMSO-d₆), δ (ppm): 1.67-1.84 (2H, m), 2.12 (3H, s), 2.76 (2H, t, J=7 Hz), 2.83-2.95 (2H, m), 3.01-3.2 (4H, m), 6.65 (2H, s), 6.78 (1H, s), 7.25 (4H, brs), 7.94 (1H, t, J=5 Hz), 12.11 (1H, brs).

MS: 352 (M+H)+ free

### Production Example 3

### Synthesis of N-(4-{2-[5-({[amino(imino)methyl]amino}methyl)-2-thienyl]ethyl}thiazol-2-yl)acetamide hydrochloride

Step 1

To a solution of N-(4-chloromethylthiazol-2-yl)acetamide (23.6 g) in toluene (200 ml) and acetonitrile (80 ml) was added triphenylphosphine (35.7 g) at 25° C. The mixture was stirred at 130° C. for 12 hours. The resulting precipitate was then collected by filtration and washed with isopropyl ether to give [(2-acetylaminothiazol-4-yl)methyl](triphenyl)phosphonium chloride (35.7 g) as colorless powder.

¹H-NMR (DMSO-d₆), δ (ppm): 2.11 (3H, s), 5.25 (2H, d, J=15.3 Hz), 6.86 (1H, d, J=3.8 Hz), 7.68-7.92 (15H, m), 12.06 (1H, s).

Step 2

N-{4-[(E)-2-(5-Formyl-2-thienyl)vinyl]thiazol-2-yl}acetamide was prepared from the compound of Step 1 of this Production Example in a manner similar to Step 2 of Production Example 1.

¹H-NMR (DMSO-d₆), δ (ppm): 2.16 (3H, s), 7.22 (1H, d, J=16 Hz), 7.35 (1H, s), 7.44 (1H, d, J=4 Hz), 7.56-7.68 (1H, m), 7.97 (1H, d, J=4 Hz), 9.88 (1H, s), 12.25 (1H, brs).

MS: 279 (M+H)

Step 3

N-{4-[2-(5-Hydroxymethyl-2-thienyl)ethyl]thiazol-2-yl}acetamide was prepared from the compound of Step 2 of this Production Example in a manner similar to Step 3 of Production Example 1.

¹H-NMR (DMSO-d₆) δ (ppm): 2.12 (3H, s), 2.9 (2H, t, J=8 Hz), 3.11 (2H, t, J=8 Hz), 4.53 (2H, d, J=6 Hz), 5.32 (1H, t, J=6 Hz), 6.65 (1H, d, J=4 Hz), 6.72 (1H, d, J=4 Hz), 6.79 (1H, s), 12.08 (1H, s).

MS: 283 (M+H)

Step 4

N-[4-(2-{5-[(1,3-Dioxo-1,3-dihydro-2H-isoindol-2-yl)methyl]-2-thienyl}ethyl)thiazol-2-yl]acetamide was prepared from the compound of Step 3 of this Production Example in a manner similar to Step 2 of Production Example 2.

¹H-NMR (DMSO-d₆), δ (ppm): 2.10 (3H, s), 2.79-3.15 (4H, m), 4.84 (2H, s), 6.67 (1H, d, J=3.5 Hz), 6.76 (1H, s), 6.87 (1H, d, J=3.5 Hz), 7.45-8.09 (4H, m), 12.06 (1H, s).

Step 5

N-{4-[2-(5-Aminomethyl-2-thienyl)ethyl]thiazol-2-yl}acetamide was prepared from the compound of Step 4 of this Production Example in a manner similar to Step 3 of Production Example 2.

¹H-NMR (DMSO-d₆), δ (ppm): 2.11 (3H, s), 2.81-3.15 (4H, m), 3.81 (2H, s), 6.64 (1H, d, J=4 Hz), 6.71 (1H, d, J=4 Hz), 6.78 (1H, s).

MS: 282 (M+H)

Step 6

Di-tert-butyl {(E)-[({5-[2-(2-acetylaminothiazol-4-yl)ethyl]-2-thienyl}methyl)amino]methylidene}biscarbamate was prepared from the compound of Step 5 of this Production Example in a manner similar to Step 4 of Production Example 2.

¹H-NMR (DMSO-d₆), δ (ppm): 1.40 (9H, s), 1.47 (9H, s), 2.19 (3H, s), 2.84-2.96 (2H, m), 3.04-3.15 (2H, m), 4.57 (2H, d, J=6 Hz), 6.67 (1H, d, J=4 Hz), 6.78 (1H, s), 6.81 (1H, d, J=4 Hz), 8.62 (1H, t, J=6 Hz), 11.45 (1H, s), 12.07 (1H, s).

MS: 524 (M+H)

Step 7

N-(4-{2-[5-({[Amino(imino)methyl]amino}methyl)-2-thienyl]ethyl}thiazol-2-yl)acetamide hydrochloride was prepared from the compound of Step 6 of this Production Example in a manner similar to Step 5 of Production Example 2.

¹H-NMR (DMSO-d₆), δ (ppm): 2.12 (3H, s), 2.90 (2H, t, J=7.3 Hz), 3.12 (2H, t, J=7.3 Hz), 4.48 (2H, d, J=6 Hz), 6.72 (1H, d, J=3.5 Hz), 6.79 (1H, s), 6.88 (1H, d, J=3.5 Hz), 7.38 (3H, brs), 8.12 (1H, t, J=6 Hz), 12.10 (1H, s).

MS: 324 (M+H)+ free

### Production Example 4

### Synthesis of 2-{5-[2-(2-acetylaminothiazol-4-yl)ethyl]-2-thienyl}-N-[amino(imino)methyl]acetamide

Step 1

To a solution of dichloromethyl methyl ether (4.97 g) in dichloromethane (50 ml) at 0° C. was added tin (IV) chloride (4.5 ml) under nitrogen atmosphere. After 15 minutes, a solution of methyl 2-thienylacetate (5 g) in dichloromethane (5 ml) was added dropwise over 30 minutes. The reaction mixture was poured into ice-water after 1 hour and then stirred for 30 minutes. The organic layer was washed with water, dried over anhydrous magnesium sulfate, and concentrated in vacuo. The residue was purified by flash column chromatography over silica gel with n-hexane/ethyl acetate (5:1→2:1) as an eluent to give methyl (5-formyl-2-thienyl)acetate (5.42 g) as yellow oil.

¹H-NMR (DMSO-d₆), δ (ppm): 3.67 (3H, s), 4.10 (2H, s), 7.19 (1H, d, J=4 Hz), 7.90 (1H, d, J=4 Hz), 9.86 (1H, s).

MS: 185 (M+H)

Step 2

Methyl {5-[(E)-2-(2-acetylaminothiazol-4-yl)vinyl]-2-thienyl}acetate was prepared from the compound of Step 1 of this Production Example in a manner similar to Step 2 of Production Example 1.

¹H-NMR (DMSO-d₆), δ (ppm): 2.14 (3H, s), 3.65 (3H, s), 3.93 (2H, s), 6.81 (1H, d, J=15.5 Hz), 6.90 (1H, d, J=4.0 Hz), 7.05 (1H, d, J=4.0 Hz), 7.15 (1H, s), 7.30 (1H, d, J=15.5 Hz), 12.19 (1H, s).

MS: 323 (M+H)+

Step 3

Methyl {5-[2-(2-acetylaminothiazol-4-yl)ethyl]-2-thienyl}acetate was prepared from the compound of Step 2 of this Production Example in a manner similar to Step 3 of Production Example 1.

¹H-NMR (DMSO-d₆), δ (ppm): 2.11 (3H, s), 2.89 (2H, t, J=7.5 Hz), 3.10 (2H, t, J=7.5 Hz), 3.62 (3H, s), 3.82 (2H, s), 6.67 (1H, d, J=3.5 Hz), 6.73 (1H, d, J=3.5 Hz), 6.78 (1H, s), 12.07 (1H, s).

MS: 325 (M+H)+

Step 4

Guanidine hydrochloride (441.7 mg) was dissolved in dimethylformamide (3 ml). To the solution was added 28% solution (0.357 ml) of sodium methoxide in methanol at room temperature. The suspension was stirred at room temperature for 30 minutes, and methyl {5-[2-(2-acetylaminothiazol-4-yl)ethyl]-2-thienyl}acetate (300 mg) was added to the mixture at room temperature. The reaction mixture was stirred at room temperature for 6 hours and concentrated in vacuo. The residue was purified by flash column chromatography over NH silica gel with trichloromethane/methanol (20:1→10:1) as an eluent. The solid was washed with acetonitrile to give 2-{5-[2-(2-acetylaminothiazol-4-yl)ethyl]-2-thienyl}-N-[amino(imino)methyl]acetamide (187.4 mg) as an off-white solid.

mp. 188.5-190° C.

¹H-NMR (DMSO-d₆), δ (ppm): 2.11 (3H, s), 2.82-2.95 (2H, m), 3.00-3.12 (2H, m), 3.50 (2H, s), 6.60 (2H, s), 6.78 (1H, s), 12.05 (1H, brs).

MS: 352 (M+H)

### Production Example 5

### Synthesis of 2-{5-[2-(2-acetylaminothiazol-4-yl)ethyl]-2-thienyl}acetic acid

2-{5-[2-(2-Acetylaminothiazol-4-yl)ethyl]-2-thienyl}acetic acid was prepared from the compound of Step 3 of Production Example 4 in a manner similar to Step 4 of Production Example 1.

mp. 172-173.5° C.

¹H-NMR (DMSO-d₆), δ (ppm): 2.11 (3H, s), 2.90 (2H, t, J=7.0 Hz), 3.10 (2H, t, J=7.0 Hz), 3.70 (2H, s), 6.66 (1H, d, J=4.0 Hz), 6.71 (1H, d, J=4.0 Hz), 6.79 (1H, s), 12.08 (1H, brs), 12.47 (1H, brs).

MS: 311 (M+H)+

### Production Example 6

### Synthesis of N-[4-(2-{5-[2-oxo-2-(1-piperazinyl)ethyl]-2-thienyl}ethyl)thiazol-2-yl]acetamide

Step 1

A mixture of 2-{5-[2-(2-acetylaminothiazol-4-yl)ethyl]-2-thienyl}acetic acid (100 mg) obtained in Production Example 5, tert-butyl 1-piperazinecarboxylate (60 mg), 1-hydroxybenzotriazole (56.6 mg) and 1-ethyl-3-(3-dimethylaminopropyl)carbodiimide hydrochloride (74.1 mg) in dichloromethane (2 ml) was stirred at room temperature for 25 hours. The reaction mixture was poured into saturated aqueous sodium hydrogen carbonate solution and extracted with trichloromethane. The organic layer was washed with water and brine, dried over anhydrous magnesium sulfate, and concentrated in vacuo. The residue was purified by preparative silica gel chromatography with trichloromethane/methanol (20:1) as an eluent to give tert-butyl 4-({5-[2-(2-acetylaminothiazol-4-yl)ethyl]-2-thienyl}acetyl)-1-piperazinecarboxylate (146.8 mg) as off-white amorphous.

¹H-NMR (DMSO-d₆), δ (ppm): 1.40 (9H, s), 2.11 (3H, s), 2.89 (2H, t, J=7.5 Hz), 3.09 (2H, t, J=7.5 Hz), 3.22-3.30 (4H, m), 3.40-3.50 (4H, m), 3.85 (2H, s), 6.64 (1H, d, J=3.5 Hz), 6.69 (1H, d, J=3.5 Hz), 6.75 (1H, s), 12.08 (1H, s).

MS: 479 (M+H)+

Step 2

tert-Butyl 4-({5-[2-(2-acetylaminothiazol-4-yl)ethyl]-2-thienyl}acetyl)-1-piperazinecarboxylate (143.2 mg), methanol (1 ml) and 4N hydrochloric acid solution (3 ml) in 1,4-dioxane were combined under nitrogen atmosphere. The reaction mixture was stirred at room temperature for 2 hours. The solvent was removed in vacuo. The residue was dissolved in water. The solution was neutralized with saturated aqueous sodium hydrogen carbonate solution and concentrated in vacuo. The residue was purified by flash column chromatography over NH silica gel with trichloromethane/methanol (10:1) as an eluent to give N-[4-(2-{5-[2-oxo-2-(1-piperazinyl)ethyl]-2-thienyl}ethyl)thiazol-2-yl]acetamide (103.4 mg) as off-white amorphous.

¹H-NMR (DMSO-d₆), δ (ppm): 2.11 (3H, s), 2.53-2.62 (4H, m), 2.89 (2H, t, J=8.0 Hz), 3.08 (2H, t, J=8.0 Hz), 3.29-3.42 (4H, m), 3.80 (2H, s), 6.64 (1H, d, J=3.5 Hz), 6.67 (1H, d, J=3.5 Hz), 6.77 (1H, s), 12.06 (1H, brs).

MS: 379 (M+H)+

### Production Example 7

### Synthesis of 2-{5-[2-(2-acetylaminothiazol-4-yl)ethyl]-2-thienyl}-N-(4-piperidinyl)acetamide

Step 1

tert-Butyl 4-[({5-[2-(2-acetylaminothiazol-4-yl)ethyl]-2-thienyl}acetyl)amino]-1-piperidinecarboxylate was prepared from the compound of Production Example 5 in a manner similar to Step 1 of Production Example 6.

¹H-NMR (DMSO-d₆), δ (ppm): 1.12-1.26 (2H, m), 1.38 (9H, s), 1.65-1.73 (2H, m), 2.11 (3H, s), 2.66-2.74 (1H, m), 2.89 (2H, t, J=7.5 Hz), 3.02-3.11 (1H, m), 3.08 (2H, t, J=7.5 Hz), 3.40-3.51 (1H, m), 3.82 (2H, s), 3.82-3.92 (1H, m), 4.15-4.23 (1H, m), 6.64 (1H, d, J=3.5 Hz), 6.67 (1H, d, J=3.5 Hz), 6.78 (1H, s), 6.87 (1H, d, J=7.5 Hz), 12.08 (1H, s).

MS: 493 (M+H)+

Step 2

2-{5-[2-(2-Acetylaminothiazol-4-yl)ethyl]-2-thienyl}-N-(4-piperidinyl)acetamide was prepared from the compound of Step 1 of this Production Example in a manner similar to Step 2 of Production Example 6.

¹H-NMR (DMSO-d₆), δ (ppm): 0.96-1.08 (2H, m), 1.59-1.69 (2H, m), 2.11 (3H, s), 2.65-2.79 (2H, m), 2.89 (2H, t, J=7.5 Hz), 2.98-3.06 (1H, m), 3.08 (2H, t, J=7.5 Hz), 3.80 (2H, s), 3.80-3.87 (1H, m), 4.10-4.18 (1H, m), 6.64 (1H, d, J=3.5 Hz), 6.67 (1H, d, J=3.5 Hz), 6.77 (1H, s).

MS: 393 (M+H)+

### Production Example 8

### Synthesis of 2-{5-[2-(2-acetylaminothiazol-4-yl)ethyl]-2-thienyl}-N-(3-pyrrolidinyl)acetamide

Step 1

tert-Butyl 3-[({5-[2-(2-acetylaminothiazol-4-yl)ethyl]-2-thienyl}acetyl)amino]-1-pyrrolidinecarboxylate was prepared from the compound of Production Example 5 in a manner similar to Step 1 of Production Example 6.

¹H-NMR (DMSO-d₆), δ (ppm): 1.39 (9H, s), 1.66-1.76 (1H, m), 1.93-2.04 (1H, m), 2.11 (3H, s), 2.88 (2H, t, J=7.5 Hz), 3.00-3.11 (1H, m), 3.08 (2H, t, J=7.5 Hz), 3.22-3.47 (3H, m), 3.52 (2H, s), 4.14 (1H, m), 6.64 (1H, d, J=4.0 Hz), 6.66 (1H, d, J=4.0 Hz), 6.78 (1H, s), 8.33 (1H, brs), 12.08 (1H, brs).

MS: 479 (M+H)+

Step 2

2-{5-[2-(2-Acetylaminothiazol-4-yl)ethyl]-2-thienyl}-N-(3-pyrrolidinyl)acetamide was prepared from the compound of Step 1 of this Production Example in a manner similar to Step 2 of Production Example 6.

¹H-NMR (DMSO-d₆), δ (ppm): 1.40-1.49 (1H, m), 1.81-1.91 (1H, m), 2.11 (3H, s), 2.43-2.49 (1H, m), 2.66-2.74 (1H, m), 2.77-2.92 (2H, m), 2.88 (2H, t, J=8.0 Hz), 3.08 (2H, t, J=8.0 Hz), 3.48 (2H, s), 4.00-4.06 (1H, m), 6.63 (1H, d, J=3.5 Hz), 6.65 (1H, d, J=3.5 Hz), 6.78 (1H, s), 8.09 (1H, d, J=4.0 Hz).

MS: 379 (M+H)+

### Production Example 9

### Synthesis of N-[4-(2-{5-[2-(3-amino-1-pyrrolidinyl)-2-oxoethyl]-2-thienyl}ethyl)thiazol-2-yl]acetamide

Step 1

tert-Butyl {[1-({5-[2-(2-acetylaminothiazol-4-yl)ethyl]-2-thienyl}acetyl)-3-pyrrolidinyl]carbamate was prepared from the compound of Production Example 5 in a manner similar to Step 1 of Production Example 6.

¹H-NMR (DMSO-d₆), δ (ppm): 1.38 (9H, s), 1.68-2.09 (2H, m), 2.11 (3H, s), 2.89 (2H, t, J=8.0 Hz), 3.08 (2H, t, J=8.0 Hz), 3.10-3.74 (6H, m), 3.90-4.07 (1H, m), 6.63-6.70 (2H, m), 6.78 (1H, s), 7.12-7.21 (1H, m), 12.08 (1H, s).

MS: 479 (M+H)+

Step 2

N-[4-(2-{5-[2-(3-Amino-1-pyrrolidinyl)-2-oxoethyl]-2-thienyl}ethyl)thiazol-2-yl]acetamide was prepared from the compound of Step 1 of this Production Example in a manner similar to Step 2 of Production Example 6.

¹H-NMR (DMSO-d₆), δ (ppm): 1.47-1.67 (1H, m), 1.82-1.98 (1H, m), 2.11 (3H, s), 2.89 (2H, t, J=8.0 Hz), 2.95-3.61 (5H, m), 3.08 (2H, t, J=8.0 Hz), 3.71 (2H, d, J=8.0 Hz), 6.64 (1H, d, J=3.5 Hz), 6.67 (1H, d, J=3.5 Hz), 6.78 (1H, s).

MS: 379 (M+H)+

### Production Example 10

### Synthesis of N-[4-(2-{4-[2-(piperazin-1-yl)ethyl]phenyl}ethyl)thiazol-2-yl]acetamide dihydrochloride

Step 1

To a solution of 2-{4-[2-(2-acetylaminothiazol-4-yl)ethyl]phenyl}acetic acid (1.07 g), obtained in Production Example 14, in dichloromethane (15 ml) was added dropwise oxalyl chloride (0.92 ml) at 5° C. After stirring for 5 minutes, 2 drops of dimethylformamide were added. The reaction mixture was stirred at 5° C. for 1 hour. After the reaction, the solvent was evaporated off. The residue was dissolved in dichloromethane (10 ml) and methanol (10 ml) under ice-cooling. This was stirred at 25° C. for 10 minutes. The organic solvent was evaporated in vacuo. The residue was dissolved into ethyl acetate. The mixture was washed with aqueous sodium hydrogen carbonate solution and brine, dried over magnesium sulfate, and filtered, and the filtrate was concentrated in vacuo. The residual yellow oil was purified by silica gel column chromatography with chloroform/methanol (20:1) as an eluent to give methyl {4-[2-(2-acetylaminothiazol-4-yl)ethyl]phenyl}acetate (970 mg) as colorless powder.

¹H-NMR (DMSO-d₆), δ (ppm): 1.98 (3H, s), 2.89 (4H, m), 3.60 (3H, s), 3.62 (2H, s), 6.73 (1H, 2), 7.19 (4H, s), 12.08 (1H, s).

MS: 319 (M+1)

Step 2

Methyl {4-[2-(2-acetylaminothiazol-4-yl)ethyl]phenyl}acetate (961 mg) was dissolved in tetrahydrofuran (14.4 ml). To the solution was added portionwise lithium tetrahydroborate (171.8 mg) at 5° C. The reaction mixture was refluxed for 4.0 hours. Sodium sulfate was added and the mixture was stirred for 12 hours. The precipitate was removed by filtration. The organic solvent was evaporated in vacuo. The residue was purified by silica gel column chromatography with n-hexane/ethyl acetate (3:2→1:1) as an eluent to give N-(4-{2-[4-(2-hydroxyethyl)phenyl]ethyl}-thiazol-2-yl)acetamide (617 mg) as powder.

¹H-NMR (DMSO-d₆), δ (ppm): 2.11 (3H, s), 2.66 (2H, t, J=7.1 Hz), 2.89 (4H, m), 3.56 (2H, m), 4.60 (1H, t, J=5.2 Hz), 6.73 (1H, s), 7.10 (4H, s), 12.07 (1H, s).

MS: 291 (M+H)

Step 3

N-(4-{2-[4-(2-Hydroxyethyl)phenyl]ethyl}thiazol-2-yl)acetamide (300 mg), carbon tetrabromide (513.9 mg), triphenylphosphine (406.5 mg) and tetrahydrofuran (3 ml) were combined at 0° C. under nitrogen atmosphere. The reaction mixture was stirred at room temperature for 1 hour, and the precipitate was filtered off. The filtrate was concentrated in vacuo. The residue was purified by flash column chromatography over silica gel with n-hexane/ethyl acetate (1:2) as an eluent. The eluate was evaporated in vacuo and the residual solid was washed with isopropyl ether to give N-(4-{2-[4-(2-bromoethyl)phenyl]ethyl}thiazol-2-yl)acetamide (227.6 mg) as an off-white solid.

mp. 153-154.5° C.

¹H-NMR (DMSO-d₆), δ (ppm): 2.11 (3H, s), 2.82-2.95 (4H, m), 3.07 (2H, t, J=7.5 Hz), 3.70 (2H, t, J=7.5 Hz), 6.73 (1H, s), 7.13 (2H, d, J=8.5 Hz), 7.18 (2H, d, J=8.5 Hz), 12.08 (1H, s).

MS: 353 (M+H)+

Step 4

N-(4-{2-[4-(2-Bromoethyl)phenyl]ethyl}thiazol-2-yl)acetamide (60 mg), tert-butyl 1-piperazinecarboxylate (40.5 mg), triethylamine (0.06 ml) and acetonitrile (1.2 ml) were combined under nitrogen atmosphere. The reaction mixture was stirred at 50° C. for 9 hours and concentrated in vacuo. The residue was purified by preparative silica gel chromatography with ethyl acetate as an eluent. The solid was washed with ethyl ether to give tert-butyl 4-(2-{4-[2-(2-acetylaminothiazol-4-yl)ethyl]phenyl}ethyl)-1-piperazinecarboxylate (25 mg) as an off-white solid.

mp. 177.5-179° C.

¹H-NMR (DMSO-d₆), δ (ppm): 1.39 (9H, s), 2.11 (3H, s), 2.34-2.39 (4H, m), 2.48 (2H, t, J=4.0 Hz), 2.68 (2H, t, J=4.0 Hz), 2.82-2.92 (4H, m), 3.27-3.32 (4H, m), 6.72 (1H, s), 7.10 (4H, s), 12.08 (1H, s).

MS: 459 (M+H)+

Step 5

N-[4-(2-{4-[2-(piperazin-1-yl)ethyl]phenyl}ethyl)thiazol-2-yl]acetamide dihydrochloride was prepared from the compound of Step 4 of this Production Example in a manner similar to Step 5 of Production Example 2.

¹H-NMR (DMSO-d₆), δ (ppm): 2.12 (3H, s), 2.83-2.95 (4H, m), 3.00-3.07 (2H, m), 3.26-3.78 (10H, m), 6.73 (1H, s), 7.18 (4H, s), 9.72 (1H, brs), 12.09 (1H, brs).

MS: 359 (M+H)+ free

### Production Example 11

### Synthesis of tert-butyl 4-{4-[2-(2-acetylaminothiazol-4-yl)ethyl]benzyl}-1-piperazinecarboxylate

Step 1

[4-(Methoxycarbonyl)benzyl](triphenyl)phosphonium bromide (6.06 g) and N,N-dimethylformamide (50 ml) were combined under nitrogen atmosphere. Potassium tert-butoxide (1.66 g) and N-(4-formylthiazol-2-yl)acetamide (2.1 g) obtained in Step 4 of Reference Example were then added to the suspension at 0° C. The reaction mixture was stirred at room temperature for 6 hours, poured into ice-water, and extracted with ethyl acetate. The organic layer was washed with 1N hydrochloric acid, water and saturated aqueous sodium chloride solution, dried over anhydrous magnesium sulfate, and concentrated in vacuo. The residue was purified by flash column chromatography over silica gel with chloroform/methanol (20:1→10:1) as an eluent and triturated with ethyl ether to give a mixture of methyl 4-[(Z)-2-(2-acetylaminothiazol-4-yl)ethenyl]benzoate and methyl 4-[(E)-2-(2-acetylaminothiazol-4-yl)ethenyl]benzoate (Z:E=3:1) (4.05 g) as a colorless solid.

mp. 164-165.5° C.

¹H-NMR (DMSO-d₆), δ (ppm): 2.13 (3H×¾, s), 2.16 (3H×¼, s), 3.85 (3H, s), 6.61 (2H×¾, s), 7.05 (1H×¾, s), 7.26 (1H×¼, d, J=15.5 Hz), 7.27 (1H×¼, s), 7.37 (1H×¼, d, J=15.5 Hz), 7.64 (2H×¾, d, J=8.5 Hz), 7.69 (2H×¼, d, J=8.5 Hz), 7.90 (2H×¾, d, J=8.5 Hz), 7.94 (2H×¼, d, J=8.5 Hz), 12.05 (1H, brs).

MS: 303 (M+H)+

Step 2

Methyl 4-[2-(2-acetylaminothiazol-4-yl)ethyl]benzoate was prepared from the compound of Step 1 of this Production Example in a manner similar to Step 2 of Production Example 9.

mp. 170-171° C.

¹H-NMR (DMSO-d₆), δ (ppm): 2.11 (3H, s), 2.86-2.95 (2H, m), 2.97-3.05 (2H, m), 3.83 (3H, s), 6.72 (1H, s), 7.35 (2H, d, J=8.5 Hz), 7.87 (2H, d, J=8.5 Hz), 12.08 (1H, brs).

MS: 305 (M+H)+

Step 3

To a stirred solution of methyl 4-[2-(2-acetylaminothiazol-4-yl)ethyl]benzoate (1.8 g) in dry tetrahydrofuran (36 ml) was added dropwise 1.0M solution of diisobutylaluminium hydride in toluene (20.7 ml) at −78° C. over 15 minutes under nitrogen atmosphere. The reaction mixture was stirred at room temperature for 1.5 hours, and the reaction was then quenched with water (1 ml). The mixture was stirred at room temperature for 30 minutes, dried over anhydrous magnesium sulfate and filtered through a Celite pad. The solvent was evaporated in vacuo. The residual solid was washed with ethyl ether to give N-(4-{2-[4-(hydroxymethyl)phenyl]ethyl}thiazol-2-yl)acetamide (1.03 g) as a colorless solid.

mp. 162-165° C.

¹H-NMR (DMSO-d₆), δ (ppm): 2.11 (3H, s), 2.80-2.95 (4H, m), 4.44 (2H, d, J=5.5 Hz), 5.09 (1H, t, J=5.5 Hz), 6.72 (1H, s), 7.14 (2H, d, J=8.0 Hz), 7.21 (2H, d, J=8.0 Hz), 12.08 (1H, brs).

MS: 277 (M+H)+

Step 4

N-(4-{2-[4-(Bromomethyl)phenyl]ethyl}thiazol-2-yl)acetamide was prepared from the compound of Step 3 of this Production Example in a manner similar to Step 3 of Production Example 10.

mp. 148.5-149.5° C.

¹H-NMR (DMSO-d₆), δ (ppm): 2.11 (3H, s), 2.82-2.98 (4H, m), 4.68 (2H, s), 6.73 (1H, s), 7.19 (2H, d, J=8.0 Hz), 7.35 (2H, d, J=8.0 Hz), 12.08 (1H, s).

MS: 339 (M+H)+

Step 5

N-(4-{2-[4-(Bromomethyl)phenyl]ethyl}thiazol-2-yl)acetamide (85.2 mg), tert-butyl 1-piperazinecarboxylate (46.8 mg), dipotassium carbonate (104.1 mg) and dimethylformamide (1.3 ml) were combined under nitrogen atmosphere. The reaction mixture was stirred at 50° C. for 4 hours. After cooling to room temperature, ethyl acetate and water were added to the mixture. The organic layer was washed with water, saturated aqueous sodium hydrogen carbonate solution and brine, dried over anhydrous magnesium sulfate, and concentrated in vacuo to give tert-butyl 4-{4-[2-(2-acetylaminothiazol-4-yl)ethyl]benzyl}-1-piperazinecarboxylate (113.1 mg) as a colorless solid.

mp. 140-141° C.

¹H-NMR (DMSO-d₆), δ (ppm): 1.38 (9H, s), 2.11 (3H, s), 2.28 (4H, t, J=5.0 Hz), 2.82-2.95 (4H, m), 3.29 (4H, t, J=5.0 Hz), 2.09 (2H, s), 6.73 (1H, s), 7.14 (2H, d, J=8.0 Hz), 7.20 (2H, d, J=8.0 Hz), 12.08 (1H, brs).

MS: 445 (M+H)+

### Production Example 12

### Synthesis of N-(4-{2-[4-(1-piperazinylmethyl)phenyl]ethyl}thiazol-2-yl)acetamide

tert-Butyl 4-{4-[2-(2-acetylaminothiazol-4-yl)ethyl]benzyl}-1-piperazinecarboxylate (95.9 mg) obtained in Production Example 11, methanol (1 ml) and 4N hydrochloric acid solution (3 ml) in 1,4-dioxane were combined under nitrogen atmosphere. The reaction mixture was stirred at room temperature for 7 hours. The solvent was removed in vacuo. The residue was dissolved in water and made basic with saturated aqueous sodium hydrogen carbonate solution. The mixture was extracted 3 times with ethyl acetate. The combined organic layer was washed with water and brine, dried over anhydrous magnesium sulfate, and concentrated in vacuo. The residual solid was washed with ethyl acetate to give N-(4-{2-[4-(1-piperazinylmethyl)-phenyl]ethyl}thiazol-2-yl)acetamide (46.9 mg) as an off-white solid.

mp. 164-165.5° C.

¹H-NMR (DMSO-d₆), δ (ppm): 2.11 (3H, s), 2.26 (4H, brs), 2.68 (4H, t, J=5.0 Hz), 2.83-2.94 (4H, m), 3.37 (2H, s), 6.73 (1H, s), 7.14 (2H, d, J=8.0 Hz), 7.18 (2H, d, J=8.0 Hz).

MS: 345 (M+H)+

### Production Example 13

### Synthesis of N-(4-{4-[2-(2-amino-1H-imidazol-5-yl)ethyl]phenyl}thiazol-2-yl)acetamide

Step 1

To a solution of ethyl 3-phenylpropanoate (8 g) in dichloromethane (25 ml) was added bromoacetyl chloride (6.0 ml). This solution was maintained under −5° C. To the solution was added aluminum chloride (16.2 g) over 15 minutes. Then, the mixture was stirred at 0° C. for 30 minutes and refluxed for 1 hour. The reaction mixture was poured into ice-water and extracted with dichloromethane. The organic layer was washed with brine, dried over magnesium sulfate, filtered, and concentrated in vacuo to give a green liquid of ethyl 3-[4-(bromoacetyl)phenyl]propanoate. This was used for the next reaction without further purification.

¹H-NMR (DMSO-d₆), δ (ppm): 1.23 (3H, t, J=7.2 Hz), 2.65 (2H, t, J=7.6 Hz), 3.02 (2H, t, J=7.6 Hz), 4.13 (2H, q, J=7.2 Hz), 4.43 (2H, s), 7.43 (2H, d, J=8.1 Hz), 7.92 (2H, d, J=8.1 Hz).

Step 2

Ethyl 3-[4-(bromoacetyl)phenyl]propanoate (13 g) was dissolved in ethanol (70 ml). To the solution was added thiourea (4.8 g), and the mixture was refluxed for 3 hours. The solution was then rotary evaporated to a reduced volume. The resulting concentrated solution was poured into water, extracted with ethyl acetate, dried over magnesium sulfate, and filtered, and the filtrate was concentrated in vacuo to give ethyl 3-[4-(2-aminothiazol-4-yl)phenyl]propanoate as pale yellow oil. This was used for the next reaction without further purification.

MS: 277 (M+H)+

Step 3

To a solution of ethyl 3-[4-(2-aminothiazol-4-yl)phenyl]propanoate (12.4 g) in dichloromethane (100 ml) were added acetyl chloride (3.82 ml) and pyridine (5.8 ml) at 25° C. This was stirred at 25° C. for 12 hours and then concentrated in vacuo. The residue was dissolved in dichloromethane and this was washed with aqueous sodium hydrogen carbonate solution and ammonium chloride. The organic layer was dried over magnesium sulfate, filtered, and concentrated in vacuo to give a brownish solid. The resulting brown solid was dissolved in methanol (80 ml) and tetrahydrofuran (50 ml). To the solution was added 1N sodium hydroxide solution (50 ml). The mixture was stirred at 25° C. for 12 hours and concentrated to a reduced volume. To the resulting aqueous solution was added 1N hydrochloric acid solution to give a colorless precipitate. This was collected by filtration and washed with water to give 3-[4-(2-acetylaminothiazol-4-yl)phenyl]propanoic acid (12.1 g) as a colorless solid.

¹H-NMR (DMSO-d₆), δ (ppm): 2.15 (3H, s), 2.52 (2H, t, J=7.5 Hz), 2.83 (2H, t, J=7.5 Hz), 7.27 (2H, d, J=8.1 Hz), 7.52 (1H, s), 7.78 (2H, d, J=8.1 Hz), 12.24 (1H, s).

MS: 291 (M+H)+

Step 4

To a solution of 3-[4-(2-acetylaminothiazol-4-yl)phenyl]propanoic acid (3 g) in dichloromethane (30 ml) was added dropwise oxalyl chloride (1.35 ml) at 5° C. After stirring for 5 minutes, 3 drops of dimethylformamide were added. The mixture was stirred at 25° C. for another 1 hour. Then, the solvent was evaporated off. The residue was dissolved in tetrahydrofuran (30 ml). To the ice-cooled solution of the resulting acid chloride was added the solution consisting of ethyl isocyanoacetate (2.82 ml), 1,8-diazabicyclo[5,4,0]-7-undecene (DBU, 4.64 ml) and tetrahydrofuran (30 ml). This was stirred at 25° C. for 2 days. This was quenched with 0.1N aqueous hydrochloric acid solution and extracted with ethyl acetate, dried over magnesium sulfate, and filtered, and the filtrate was concentrated in vacuo. The residue was purified by silica gel column chromatography with dichloromethane/methanol (5:1) as an eluent to give ethyl 5-{2-[4-(2-acetylaminothiazol-4-yl)phenyl]ethyl}-1,3-oxazole-4-carboxylate (2.25 g) as white powder.

¹H-NMR (DMSO-d₆), δ (ppm): 1.26 (3H, t, J=7.1 Hz), 2.15 (3H, s), 2.96 (2H, t, J=7.4 Hz), 3.33 (2H, t, J=7.4 Hz), 4.22 (2H, q, J=7.1 Hz), 7.21 (2H, d, J=8.2 Hz), 7.54 (1H, s), 7.78 (2H, d), 8.37 (1H, s), 12.20 (1H, s).

MS: 386 (M+H)+

Step 5

To a solution of ethyl 5-{2-[4-(2-acetylaminothiazol-4-yl)phenyl]ethyl}-1,3-oxazole-4-carboxylate (2.14 g) in methanol (5 ml) was added conc. hydrochloric acid (10 ml). The mixture was stirred at 80° C. for 8 hours and concentrated in vacuo to give 1-amino-4-[4-(2-aminothiazol-4-yl)phenyl]-2-butanone dihydrochloride as a crude solid. This was used for the next reaction without further purification.

¹H-NMR (DMSO-d₆), δ (ppm): 2.93 (4H, m), 3.95 (2H, m), 7.02 (1H, s), 7.33 (2H, d, J=8.4 Hz), 7.63 (2H, d, J=8.4 Hz), 8.21 (3H, br), 8.77 (2H, br).

Step 6

To a solution of 1-amino-4-[4-(2-aminothiazol-4-yl)phenyl]-2-butanone dihydrochloride (1.8 g) in methylene chloride (20 ml) and dimethylformamide (20 ml) were added diisopropylethylamine (3.3 ml) and di-tert-butyl dicarbamate (1.29 g). The mixture was stirred at room temperature for 12 hours. To the mixture was added water and the solution was extracted with dichloromethane. The organic layer was dried over magnesium sulfate, filtered, and concentrated. The residual oil was dissolved in pyridine (20 ml). To this solution was added acetyl chloride (0.57 ml) under ice-cooling. The mixture was stirred at 25° C. for 2 hours. The solution was then poured into water, and the organic layer was extracted with ethyl acetate. After drying over magnesium sulfate, the solvent was removed in vacuo and the residual yellow oil was purified by silica gel column chromatography with n-hexane/ethyl acetate (10:1) as an eluent to give tert-butyl {4-[4-(2-acetylaminothiazol-4-yl)phenyl]-2-oxobutyl}carbamate (0.3 g) as white powder.

¹H-NMR (DMSO-d₆), δ (ppm): 1.38 (9H, s), 2.15 (3H, s), 2.76 (4H, br), 3.76 (2H, d, J=5.8 Hz), 7.07 (1H, t), 7.25 (2H, d, J=8.2 Hz), 7.53 (1H, s), 7.78 (2H, d), 12.22 (1H, s).

MS: 404 (M+H)+

Step 7

tert-Butyl {4-[4-(2-acetylaminothiazol-4-yl)phenyl]-2-oxobutyl}carbamate (260 mg) was treated with 4N hydrochloric acid in dioxane at room temperature for 2 hours. Then the solvent was evaporated in vacuo. The residue was triturated with isopropyl ether to give N-{4-[4-(4-amino-3-oxobutyl)phenyl]thiazol-2-yl}acetamide hydrochloride (218 mg) as colorless powder.

¹H-NMR (DMSO-d₆), δ (ppm): 2.16 (3H, s), 2.89 (4H, d×2), 3.81 (2H, m), 7.28 (2H, d, J=8.2 Hz), 7.54 (1H, s), 7.80 (2H, d, J=8.2 Hz), 8.10 (3H, br), 12.23 (1H, br).

MS: 304 (M+H)+

Step 8

To a solution of N-{4-[4-(4-amino-3-oxobutyl)phenyl]thiazol-2-yl}acetamide hydrochloride (100 mg) in water (2 ml) was added 1N sodium hydroxide solution to adjust the pH to 4.5. To this solution was added cyanamide (62 mg) and the mixture was stirred at. 100° C. for 3 hours. The reaction mixture was cooled to room temperature, and 1N aqueous sodium hydroxide solution was added. The mixture was extracted with tetrahydrofuran. The organic layer was dried over magnesium sulfate, filtered, and concentrated in vacuo to give yellow oil. The residue was triturated with isopropyl ether and ethanol to give N-(4-{4-[2-(2-amino-1H-imidazol-5-yl)ethyl]phenyl}thiazol-2-yl)acetamide (79 mg) as white powder.

¹H-NMR (DMSO-d₆), δ (ppm): 2.15 (3H, s), 3.15 (2H, s), 3.17 (2H, s), 4.15 (2H, d, J=2.5 Hz), 6.71 (2H, br), 7.25 (2H, br), 7.78 (2H, br), 12.20 (1H, s).

MS: 328 (M+H)+

### Production Example 14

### Synthesis of 2-{4-[2-(2-acetylaminothiazol-4-yl)ethyl]phenyl}acetic acid

Step 1

To a solution of [4-(bromomethyl)phenyl]acetic acid (5.0 g) in toluene (50 ml) was added triphenylphosphine (5.8 g) at 25° C. This was refluxed for 5 hours. After cooling to room temperature, the resulting colorless precipitate was collected by filtration and washed with isopropyl ether to give [4-(carboxymethyl)benzyl](triphenyl)phosphonium bromide (10.7 g) as white powder.

¹H-NMR (DMSO-d₆), δ (ppm): 3.52 (2H, s), 5.13 (2H, d, J=15.6 Hz), 6.90 (2H, dd, J=8.1, 2.3 Hz), 7.11 (2H, d, J=8.1 Hz), 7.58-7.91 (15H, m).

MS: 411 (M+H)+

Step 2

To a solution of [4-(carboxymethyl)benzyl](triphenyl)-phosphonium bromide (19.1 g) in dimethylformamide (180 ml) was added potassium tert-butoxide (11.9 g) under ice-cooling. This was stirred at 5° C. for 30 minutes. To the solution was added N-(4-formylthiazol-2-yl)acetamide (6.0 g) obtained in Step 4 of Reference Example in dimethylformamide (18 ml). This was stirred at 25° C. for 3 hours. The mixture was poured into water and extracted with ethyl acetate. The aqueous layer was acidified to pH 4-5 with 1N hydrochloric acid to give a colorless precipitate. The precipitate was collected by filtration to give a mixture of 2-{4-[(E)-2-(2-acetylaminothiazol-4-yl)vinyl]phenyl}acetic acid and 2-{4-[(Z)-2-(2-acetylaminothiazol-4-yl)vinyl]phenyl}acetic acid (10 g) as white powder.

¹H-NMR (DMSO-d₆), δ (ppm): 2.12-2.14 (3×⅚, 3×⅙H, s), 3.52-3.54 (2×⅚, 2×⅙H, s), 6.46 (⅚H, d, J=12.7 Hz), 6.54 (⅚H, d, J=12.7 Hz), 6.95 (1H, s), 7.11-7.49 (4+⅙H, m), 12.09 (1H, br).

MS: 303 (M+H)+

Step 3

2-{4-[2-(2-Acetylaminothiazol-4-yl)ethyl]phenyl}acetic acid was prepared from the compound of Step 2 of this Production Example in a manner similar to Step 3 of Production Example 1.

¹H-NMR (DMSO-d₆), δ (ppm): 2.11 (3H, s), 2.88 (4H, s), 3.50 (2H, s), 6.74 (1H, s), 7.14 (4H, s), 12.08 (1H, s).

MS: 305 (M+H)

### Production Example 15

### Synthesis of 2-{4-[2-(2-acetylaminothiazol-4-yl)ethyl]phenyl}-N-1H-imidazol-2-ylacetamide

To a solution of 2-{4-[2-(2-acetylaminothiazol-4-yl)ethyl]phenyl}acetic acid (140 mg) obtained in Production Example 14 in dimethylformamide (3.0 ml) were added 2-aminoimidazole sulfate (122 mg), benzotriazol-1-yloxytris(dimethylamino)phosphonium hexafluorophosphate (264 mg) and diisopropylethylamine (0.10 ml) at 25° C. The mixture was stirred at 70° C. for 15 hours and poured into water. The resulting precipitate was collected by filtration and triturated with isopropyl ether and isopropyl alcohol to give 2-{4-[2-(2-acetylaminothiazol-4-yl)ethyl]phenyl}-N-1H-imidazol-2-ylacetamide (75.7 mg) as pale yellow powder.

¹H-NMR (DMSO-d₆), δ (ppm): 2.11 (3H, s), 2.88 (4H, m), 3.49 (2H, s), 6.74 (1H, s), 7.14 (6H, s), 12.09 (2H, br).

MS: 370 (M+H)

### Production Example 16

### Synthesis of N-[4-(2-{4-[2-(2-amino-1H-imidazol-1-yl)-2-oxoethyl]phenyl}ethyl)thiazol-2-yl]acetamide

To a solution of 2-{4-[2-(2-acetylaminothiazol-4-yl)ethyl]phenyl}acetic acid (150 mg) obtained in Production Example 14 in dimethylformamide (3.0 ml) was added 1-hydroxybenzotriazole (99.9 mg), and the mixture was cooled in an ice-bath. To the solution was added portionwise 1-(3-dimethylaminopropyl)-3-ethylcarbodiimide methioide (292 mg). The reaction mixture was allowed to room temperature and stirred for 2.5 hours. To the reaction mixture was added a solution of 2-aminoimidazole sulfate (130 mg) and diisopropylethylamine (0.18 ml) in dimethylformamide (1 ml). The mixture was stirred at 25° C. for 24 hours and then at 55° C. for 12 hours. The mixture was poured into water. The resulting precipitate was collected by filtration and dissolved in 1N hydrochloric acid. The aqueous layer was washed with ethyl acetate. To the aqueous layer was added 1N sodium hydroxide solution to give a precipitate. The precipitate was collected by filtration and washed with isopropyl ether to give N-[4-(2-{4-[2-(2-amino-1H-imidazol-1-yl)-2-oxoethyl]phenyl}ethyl)thiazol-2-yl]acetamide (29.6 mg) as white powder.

¹H-NMR (DMSO-d₆), δ (ppm): 2.10 (3H, s), 2.10 (3H, s), 2.85-2.90 (4H, m), 3.59 (2H, s), 6.64 (1H, s), 6.73 (2H, s×2), 7.14 (2H, d, J=8.0 Hz), 7.22 (2H, d, J=8.0 Hz), 11.28 (1H, s), 44.49 (1H, s), 12.08 (1H, s).

MS: 370 (M+H)

### Production Example 17

### Synthesis of N-[4-(2-{4-[(2-amino-1H-imidazol-4-yl)methyl]phenyl}ethyl)thiazol-2-yl]acetamide

Step 1

To a solution of 2-{4-[2-(2-acetylaminothiazol-4-yl)ethyl]phenyl}acetic acid (300 mg) in dichloromethane (4.5 ml) was added dropwise oxalyl chloride (0.15 ml) at 5° C. After stirring for 5 minutes, 2 drops of dimethylformamide were added. The reaction mixture was stirred under ice-cooling for 0.5 hour. Then, the solvent was evaporated to give yellow-green powder. This acid chloride was dissolved in dichloromethane (4.5 ml) and the solution was cooled in an ice-bath. To the solution was added dropwise (trimethylsilyl)diazomethane (2M in hexane). The reaction mixture was stirred at 5° C. for 45 minutes. The solvent was removed in vacuo to give brownish oil. This diazoketone was dissolved in dichloromethane (4.5 ml), and the solution was ice-cooled. To the solution was added hydrobromic acid (33% in acetic acid, 0.11 ml). After stirring at 55° C. for 45 minutes, aqueous sodium hydrogen carbonate solution was added to the solution. The mixture was extracted with tetrahydrofuran, washed with brine, dried over magnesium sulfate, and filtered, and the filtrate was concentrated in vacuo to give N-(4-{2-[4-(3-bromo-2-oxopropyl)phenyl]ethyl}thiazol-2-yl)acetamide. This was used for the next reaction without further purification.

MS: 381, 383 (M+H)+

Step 2

To the crude oil of N-(4-{2-[4-(3-bromo-2-oxopropyl)phenyl]ethyl}thiazol-2-yl)acetamide (375 mg) in dimethylformamide (4.5 ml) was added sodium azide (128 mg) under ice-cooling. The mixture was stirred at 5° C. for 2 hours and poured into water. The mixture was extracted with ethyl acetate, washed with brine, dried over magnesium sulfate, and filtered, and the filtrate was concentrated in vacuo. The residue was purified by silica gel column chromatography with n-hexane/ethyl acetate (1:1→1:2) to give N-(4-{2-[4-(3-azido-2-oxopropyl)phenyl]ethyl}thiazol-2-yl)acetamide (64 mg) as white powder.

¹H-NMR (DMSO-d₆), δ (ppm): 2.11 (3H, s), 2.83-2.88 (4H, m), 3.74 (2H, s), 4.26 (2H, s), 6.73 (1H, s), 7.10 (2H, d, J=8.2 Hz), 7.16 (2H, d, J=8.2 Hz), 12.02 (1H, s, J=1.0 Hz).

MS: 344 (M+H)+

The resulting N-(4-{2-[4-(3-azido-2-oxopropyl)phenyl]ethyl}thiazol-2-yl)acetamide (64 mg) was dissolved in methanol (2 ml) and tetrahydrofuran (2 ml). To the solution were added 1N hydrochloric acid (0.563 ml) and 10% palladium on carbon (50% wet, 50 mg). The mixture was stirred under 3 atm hydrogen pressure for 2 hours and filtered through a Celite pad. The filtrate was concentrated in vacuo to give N-(4-{2-[4-(3-amino-2-oxopropyl)phenyl]ethyl}thiazol-2-yl)acetamide hydrochloride.

MS: 318 (M+H)+ free

This was used for the next reaction without further purification.

Step 3

To a solution of N-(4-{2-[4-(3-amino-2-oxopropyl)phenyl]ethyl}thiazol-2-yl)acetamide hydrochloride (64 mg) in water (5 ml) was added 1N sodium hydroxide solution to adjust the pH to 4.5. To this solution was added cyanamide (76 mg) at room temperature. The reaction mixture was stirred at 100° C. for 2 hours. The reaction mixture was cooled, and 1N hydrochloric acid was added to the reaction mixture. The mixture was washed with ethyl acetate. To the aqueous layer was added 1N sodium hydroxide solution under stirring to give a colorless precipitate. Filtration of the resulting precipitate gave N-[4-(2-{4-[(2-amino-1H-imidazol-4-yl)methyl]phenyl}ethyl)thiazol-2-yl]acetamide (25 mg) as white powder.

¹H-NMR (DMSO-d₆), δ (ppm): 2.10 (3H, s), 2.82-2.89 (4H, m), 3.55 (2H, s), 4.93 (2H, s), 6.10 (1H, s), 6.57 (0.6H, s), 6.72 (1H, s), 7.07 (2H, d, J=8.0 Hz), 7.10 (2H, d, J=8.0 Hz), 9.90 (0.4H, s), 12.08 (1H, s).

MS: 342 (M+H)+

### Production Example 18

### Synthesis of N-[4-(2-{4-[(2-aminothiazol-4-yl)methyl]phenyl}ethyl)thiazol-2-yl]acetamide

The crude oil of N-(4-{2-[4-(3-bromo-2-oxopropyl)phenyl]ethyl}thiazol-2-yl)acetamide (175 mg) obtained in Step 1 of Production Example 17 was dissolved in ethanol (3 ml). To the solution was added thiourea (35 mg), and the mixture was stirred at 50° C. for 2.5 hours. The organic solvent was evaporated to a reduced volume. To the resulting solution was added aqueous sodium hydrogen carbonate solution. The mixture was extracted with tetrahydrofuran, and the organic layer was washed with brine, dried over magnesium sulfate, and filtered, and the filtrate was concentrated in vacuo. The residue was purified by silica gel column chromatography with chloroform/methanol (10:1) to give N-[4-(2-{4-[(2-aminothiazol-4-yl)methyl]phenyl}ethyl)thiazol-2-yl]acetamide (10 mg) as pale yellow powder.

¹H-NMR (DMSO-d₆), δ (ppm): 2.11 (3H, s), 2.86 (4H, br), 3.66 (2H, br), 6.10 (1H, s), 6.73 (1H, s), 6.80 (2H, s), 7.10 (4H, s), 12.07 (1H, s).

MS: 359 (M+H)

### Production Example 19

### Synthesis of N-[4-(2-{4-[2-(2-amino-1H-imidazol-1-yl)ethyl]phenyl}ethyl)thiazol-2-yl]acetamide

### Preparation of methyl [4-{2-(2-acetylaminothiazol-4-yl)ethyl}phenyl]acetate

To a solution of 2-{4-[2-(2-acetylaminothiazol-4-yl)ethyl]phenyl}acetic acid (1.07 g) in CH₂Cl₂ (15 ml) was added oxalyl chloride (0.92 ml) dropwise at 5° C. After 5 minutes stirring, 2 drops of DMF was added. The reaction mixture was stirred at 5° C. for 1 hr. After the reaction, the solvent and reagents were evaporated. The residue was dissolved in CH₂Cl₂ (10 ml) and MeOH (10 ml) under ice-cooling. This was stirred at 25° C. for 10 min. The organic solvent was evaporated in vacuo. The residue was dissolved in ethyl acetate. The mixture was washed with aq. NaHCO₃ solution and brine, subsequently. The combined organic layer was dried over MgSO₄ and filtered, and the filtrate was concentrated in vacuo. The residual yellow oil was purified by silicagel column chromatography with chloroform and Methanol (20/1) as an eluent to give methyl [4-{2-(2-acetylaminothiazol-4-yl)ethyl}phenyl]acetate (970 mg) as colourless powder.

¹H-NMR (DMSO-d₆), δ (ppm): 1.98 (3H, s), 2.89 (4H, m), 3.60 (3H, s), 3.62 (2H, s), 6.73 (1H, s), 7.19 (4H, s), 12.08 (1H, s).

ESI m/z 319 (M+1)

### Preparation of N-(4-{2-[4-(2-hydroxyethyl)phenyl]ethyl}-thiazol-2-yl)acetamide

N-(4-{2-[4-(2-hydroxyethyl)phenyl]ethyl}-thiazol-2-yl)acetamide was prepared from methyl [4-{2-(2-acetylaminothiazol-4-yl)ethyl}phenyl]acetate.

¹H-NMR (DMSO-d₆), δ (ppm): 2.11 (3H, s), 2.66 (2H, t, J=7.1 Hz), 2.89 (4H, m), 3.56 (2H, m), 4.60 (1H, t, J=5.2 Hz), 6.73 (1H, s), 7.10 (4H, s), 12.07 (1H, s).

m/z 291 (M+H)

Step 1

To a suspension of N-(4-{2-[4-(2-hydroxyethyl)phenyl]-ethyl}thiazol-2-yl)acetamide (145 mg) obtained in Step 2 of Production Example 10 in tetrahydrofuran (2.9 ml) were added triphenylphosphine (157 mg) and carbon tetrabromide (249 mg) under ice-cooling. The mixture was stirred at 25° C. for 1 hour and concentrated. The residue was purified by silica gel column chromatography with hexane and ethyl acetate as an eluent to give white powder. A solution of the resulting white powder in dimethylformamide (3 ml) was added dropwise to a solution of aminoacetaldehyde dimethylacetal (525 mg) in dimethylformamide (3 ml) under ice-cooling. The solution was stirred at 65° C. for 1.5 hours and poured into water. The mixture was extracted with ethyl acetate. The organic layer was washed with brine, dried over magnesium sulfate, and filtered, and the filtrate was concentrated in vacuo to give crude N-{4-[2-(4-{2-[(2,2-dimethoxyethyl)amino]ethyl}-phenyl)ethyl]thiazol-2-yl}acetamide as colorless oil. This was used for the next reaction without further purification.

MS: 378 (M+H)

Step 2

Di-tert-butyl {(Z)-[(2-{4-[2-(2-acetylaminothiazol-4-yl)ethyl]phenyl}ethyl)(2,2-dimethoxyethyl)amino]-methylidene}biscarbamate was prepared from the compound of Step 1 of this Production Example in a manner similar to Step 4 of Production Example 2.

¹H-NMR (DMSO-d₆), δ (ppm): 1.37 (9H, s), 1.41 (9H, s), 2.11 (3H, s), 2.67-2.80 (2H, m), 2.87 (5H, m), 3.27 (8H, s×2), 3.50 (2H, m), 4.38 (2H, m), 6.71 (1H, s), 7.11 (4H, s), 12.07 (1H, s).

MS: 620 (M+H)

Step 3

N-[4-(2-{4-[2-(2-Amino-1H-imidazol-1-yl)ethyl]phenyl}ethyl)thiazol-2-yl]acetamide was prepared from the compound of Step 2 of this Production Example in a manner similar to Step 7 of Production Example 1.

¹H-NMR (DMSO-d₆), δ (ppm): 2.11 (3H, s), 2.81-2.87 (6H, m), 3.85 (2H, dd, J=8.03, 6.53 Hz), 5.21 (2H, s), 6.31 (1H, d, J=1.51 Hz), 6.50 (1H, d, J=1.51 Hz), 6.71 (1H, s), 7.10 (2H, d, J=8.03 Hz), 7.18 (2H, d, J=8.03 Hz), 12.11 (1H, br).

MS: 356 (M+H)

### Production Example 20

### Synthesis of N-{4-[2-(2-{[amino(imino)methyl]amino}-1H-benzimidazol-6-yl)ethyl]thiazol-2-yl}acetamide

Step 1

To a suspension of (3,4-dinitrobenzyl)(triphenyl)-phosphonium bromide (1.54 g) in N,N-dimethylformamide (5 ml) was added potassium tert-butoxide (363 mg) at 0° C. under nitrogen atmosphere. The mixture was stirred at 0° C. for 10 minutes, and N-(4-formylthiazol-2-yl)acetamide (500 mg) was added to the mixture at 0° C. The reaction mixture was stirred at 20° C. for 4 hours. Ethyl acetate (50 ml) was added, and the mixture was washed with water (20 ml×3) and brine. The organic layer was dried over magnesium sulfate and evaporated to give a crude yellow foam (1.62 g). The crude material was purified by flash column chromatography over silica gel with chloroform/ethyl acetate (1:1) as an eluent to give a mixture of N-{4-[(Z)-2-(3,4-dinitrophenyl)vinyl]thiazol-2-yl}acetamide and N-{4-[(E)-2-(3,4-dinitrophenyl)vinyl]thiazol-2-yl}acetamide (Z:E=8:1; 864.7 mg) as an orange foam.

¹H-NMR (DMSO-d₆), δ (ppm): 2.13 (3H× 8/9, s), 2.17 (3H× 1/9, s), 6.64 (1H× 8/9, d, J=12.6 Hz), 6.80 (1H× 8/9, d, J=12.6 Hz), 7.29 (1H× 1/9, d, J=15.7 Hz), 7.33 (1H× 8/9, s), 7.39 (1H× 1/9, s), 7.63 (1H× 1/9, d, J=15.7 Hz), 8.00-8.50 (3H, m), 11.97 (1H× 8/9, s), 12.30 (1H× 1/9, s).

MS: 335.0 (M+H)+, 357.1 (M+Na)+

Step 2

A mixture of N-{4-[(Z)-2-(3,4-dinitrophenyl)vinyl]thiazol-2-yl}acetamide and N-{4-[(E)-2-(3,4-dinitrophenyl)vinyl]thiazol-2-yl}acetamide (Z:E=8:1) (653 mg), methanol (7.6 ml), tetrahydrofuran (5 ml), acetic acid (0.26 ml) and 10% palladium on carbon (684 mg) were sequentially combined under nitrogen atmosphere. The mixture was stirred at ambient temperature under 3 atm hydrogen pressure for 2 days. The reaction mixture was filtered through a Celite pad, and the filtrate was concentrated in vacuo. The residue was dissolved in ethyl acetate. The organic solution was washed with saturated sodium hydrogen carbonate solution and saturated sodium chloride solution, dried over magnesium sulfate, and evaporated to give a crude material (658.1 mg). The crude material was purified by flash column chromatography over NH-silica gel with chloroform/methanol (100:0->100:1) as an eluent to give N-{4-[2-(3,4-diaminophenyl)ethyl]thiazol-2-yl}acetamide (499.8 mg) as pale brown amorphous.

¹H-NMR (CDCl₃), δ (ppm): 2.22 (3H, s), 2.58-3.17 (8H, m), 6.46-6.56 (3H, m), 6.62 (1H, d, J=8.3 Hz), 8.84-10.42 (1H, brs).

MS: 277.1 (M+H)+, 299.2 (M+Na)+

Step 3

N-{4-[2-(3,4-Diaminophenyl)ethyl]thiazol-2-yl}acetamide (100 mg) was treated with 10% hydrochloric acid in methanol (2 ml) at 0° C. The volatiles were evaporated in vacuo. To a suspension of the residue in 2-propanol (0.831 ml) was added dicyandiamide (45.6 mg), and the mixture was heated under reflux for 20 hours. The solvent was removed in vacuo, and to the residue was added ethyl acetate (10 ml). The resulting precipitate was removed by filtration. The filtrate was evaporated and solidified with dichloromethane (10 ml) to give N-{4-[2-(2-{[amino(imino)methyl]amino}-1H-benzimidazol-6-yl)ethyl]thiazol-2-yl}acetamide (28.0 mg) as a white solid.

¹H-NMR (DMSO-d₆+D₂O), δ (ppm): 2.12 (3H, s), 2.81-2.99 (4H, m), 6.69-6.81 (2H, m), 6.92-7.09 (2H, m).

MS: 344.14 (M+H)+

### Production Example 21

### Synthesis of N-(4-{2-[4-(2-ureidoethyl)phenyl]ethyl}thiazol-2-yl)acetamide

Step 1

To a solution of phthalimide potassium salt (46.2 g) in N,N-dimethylformamide (300 ml) was added dropwise 4-(2-bromoethyl)benzaldehyde (40.92 g) in N,N-dimethylformamide (50 ml) at 60° C., and the mixture was stirred for 2 hours. The reaction mixture was cooled to 20° C. and poured into water (1.5 L). The resulting precipitate was collected by filtration to give a yellow solid. The solid was dissolved in chloroform (250 ml), and the insoluble material was removed by filtration. The filtrate was concentrated in vacuo. The residue was washed with diethyl ether and collected by filtration to give 4-[2-(1,3-dioxo-1,3-dihydro-2H-isoindol-2-yl)ethyl]benzaldehyde (19.65 g) as a off-white solid.

¹H-NMR (DMSO-d₆), δ (ppm): 3.04 (2H, t, J=7 Hz), 3.88 (2H, t, J=7 Hz), 7.44 (2H, d, J=8.5 Hz), 7.75-7.89 (6H, m), 9.94 (1H, s).

MS: 280.1 (M+H)+

Step 2

{[2-Acetylaminothiazol-4-yl]methyl}(triphenyl)phosphonium chloride (46.9 mg) and dimethylformamide (190 ml) were combined under nitrogen atmosphere, and potassium tert-butoxide (12.8 g) was then added to the suspension at 0° C. The reaction mixture was stirred at 0° C. for 15 minutes, and 4-[2-(1,3-dioxo-1,3-dihydro-2H-isoindol-2-yl)ethyl]benzaldehyde (19.28 g) was added to the mixture at 0° C. The reaction mixture was stirred at 20° C. for 2 hours. The reaction mixture was poured into water, and the resulting precipitate was collected by filtration to give a crude brown solid. The brown solid was washed with acetonitrile/isopropyl ether (1:1)→acetonitrile to give N-[4-((E)-2-{4-[2-(1,3-dioxo-1,3-dihydro-2H-isoindol-2-yl)ethyl]phenyl}vinyl)thiazol-2-yl]acetamide (24.88 g) as a beige solid.

¹H-NMR (DMSO-d₆), δ (ppm): 2.15 (3H, s), 2.94 (2H, t, J=7.1 Hz), 3.83 (2H, t, J=7.1 Hz), 7.12 (1H, d, J=15.8 Hz), 7.14 (1H, d, J=15.8 Hz), 7.16 (1H, s), 7.19 (2H, d, J=8 Hz), 7.44 (2H, d, J=8.4 Hz), 7.8-7.88 (4H, m), 12.22 (1H, s).

MS: 418.1 (M+H)+

Step 3

N-[4-((E)-2-{4-[2-(1,3-Dioxo-1,3-dihydro-2H-isoindol-2-yl)ethyl]phenyl}vinyl)thiazol-2-yl]acetamide (24.88 g), dimethylformamide (800 ml), methanol (80 ml), acetic acid (8 ml) and 10% palladium on carbon (50% wet, 24.4 g) were combined in sequence under nitrogen atmosphere. The mixture was stirred at 20° C. under hydrogen atmosphere (4 atm) for 16 hours. The catalyst was renewed every 4 hours during the reaction. The reaction mixture was filtered through a Celite pad, and the filtrate was concentrated in vacuo. The residue was washed with isopropyl ether (200 ml) and purified by flash column chromatography over silica gel with trichloromethane/ethyl acetate (1:1) as an eluent. The fractions containing the object compound were combined and evaporated in vacuo. The residue was washed with isopropyl ether (200 ml) and collected by filtration to give N-[4-(2-{4-[2-(1,3-dioxo-1,3-dihydro-2H-isoindol-2-yl)ethyl]phenyl}ethyl)thiazol-2-yl]acetamide (17.86 g) as an off-white solid.

¹H-NMR (DMSO-d₆), δ (ppm): 2.11 (3H, s), 2.78-2.92 (6H, m), 3.79 (2H, t, J=7.3 Hz), 6.66 (1H, s), 7.08 (2H, d, J=8.9 Hz), 7.1 (2H, d, J=8.8 Hz), 7.79-7.89 (4H, m), 12.08 (1H, s).

MS: 420.2 (M+H)+, 442.1 (M+Na)+

Step 4

To a solution of N-[4-(2-{4-[2-(1,3-dioxo-1,3-dihydro-2H-isoindol-2-yl)ethyl]phenyl}ethyl)thiazol-2-yl]acetamide (2.06 g) in acetonitrile (20 ml) was added hydrazine monohydrate (2.38 ml), and the mixture was stirred at 50° C. for 2 hours. The volatiles were evaporated. To the mixture was added chloroform (10 ml) and the insoluble material was removed by filtration to give a crude pale yellow foam. The crude foam was purified by flash column chromatography over NH₂-silica gel with trichloromethane/methanol (10:0→10:2) as an eluent to give N-(4-{2-[4-(2-aminoethyl)phenyl]ethyl}-thiazol-2-yl)acetamide (1.1304 g) as a pale yellow solid.

¹H-NMR (DMSO-d₆), δ (ppm): 2.11 (3H, s), 2.58 (2H, t, J=7.3 Hz), 2.72 (2H, t, J=7.1 Hz), 2.81-2.94 (4H, m), 6.73 (1H, s), 7.08 (2H, d, J=8.4 Hz), 7.11 (2H, d, J=8.4 Hz).

MS: 290.2 (M+H)+

Step 5

To a suspension of N-(4-{2-[4-(2-aminoethyl)phenyl]ethyl}thiazol-2-yl)acetamide (100 mg) in trichloromethane (1 ml) were added N,N-diisopropylethylamine (90.3 μl) and isocyanato(trimethyl)silane (93.6 μl), and the mixture was stirred at 20° C. for 18 hours. The resulting precipitate was collected by filtration and washed with trichloromethane/methanol (10:1) to give N-(4-{2-[4-(2-ureidoethyl)phenyl]ethyl}thiazol-2-yl)acetamide (49.6 mg) as a white solid.

¹H-NMR (DMSO-d₆), δ (ppm): 2.11 (3H, s), 2.62 (2H, t, J=7.3 Hz), 2.82-2.94 (4H, m), 3.16 (2H, q, J=6.8 Hz), 5.42 (2H, s), 5.89 (1H, t, J=5.7 Hz), 6.74 (1H, s), 7.1 (2H, d, J=8 Hz), 7.13 (2H, d, J=8 Hz), 11.73 (1H, brs).

MS: 333.3 (M+H)+, 355.1 (M+Na)+

### Production Example 22

### Synthesis of N-{4-[2-(4-{2-[(methanesulfonyl)amino]ethyl}phenyl)ethyl]thiazol-2-yl}acetamide

To a suspension of N-(4-{2-[4-(2-aminoethyl)phenyl]ethyl}thiazol-2-yl)acetamide (100 mg) obtained in Step 4 of Production Example 21 in trichloromethane (1 ml) were added N,N-diisopropylethylamine (180.6 μl) and methanesulfonyl chloride (53.5 μl) at 0° C., and the mixture was stirred for 3 hours. The reaction mixture was added to water (1 ml). The resulting precipitate was collected by filtration and washed with dichloromethane to give N-{4-[2-(4-{2-[(methanesulfonyl)amino]ethyl}phenyl)ethyl]thiazol-2-yl}acetamide (66.3 mg) as a white solid.

¹H-NMR (DMSO-d₆), δ (ppm): 2.11 (3H, s), 2.72 (2H, t, J=7.7 Hz), 2.81 (3H, s), 2.82-2.94 (4H, m), 3.1-3.18 (2H, m), 6.73 (1H, s), 7.07 (1H, t, J=5.8 Hz), 7.14 (4H, s), 12.08 (1H, s).

MS: 368.2 (M+H)+, 390.1 (M+Na)+

### Production Example 23

### Synthesis of N-{[(2-{4-[2-(2-acetylaminothiazol-4-yl)ethyl]phenyl}ethyl)amino]-carbonothioyl}benzamide

N-(4-{2-[4-(2-Aminoethyl)phenyl]ethyl}thiazol-2-yl)acetamide (200 mg) obtained in Step 4 of Production Example 21 was dissolved in acetone (2.8 ml) under nitrogen atmosphere, and then isothiocyanic acid benzoyl ester (93.2 μl) was added dropwise to the solution at 0° C. The reaction mixture was stirred at 20° C. for 1 hour. Water was added to the mixture, and the precipitate was filtered in vacuo to give a crude yellow solid. The crude solid was purified by flash column chromatography over silica gel with trichloromethane/methanol (100:0→100:2) as an eluent to give N-{[(2-{4-[2-(2-acetylaminothiazol-4-yl)ethyl]phenyl}ethyl)amino]carbonothioyl}benzamide (152.8 mg) as a pale yellow solid.

¹H-NMR (DMSO-d₆), δ (ppm): 2.11 (3H, s), 2.81-2.96 (6H, m), 3.82 (2H, q, J=6.7 Hz), 6.72 (1H, s), 7.15 (2H, d, J=8 Hz), 7.19 (2H, d, J=8 Hz), 7.51 (2H, t, J=7.7 Hz), 7.63 (1H, t, J=7.5 Hz), 7.91 (2H, d, J=7.7 Hz), 10.93 (1H, t, J=5.3 Hz), 11.34 (1H, s), 12.09 (1H, s).

MS: 453.3 (M+H)+, 475.1 (M+Na)+

### Production Example 24

### Synthesis of N-(4-{2-[4-(2-thioureidoethyl)phenyl]ethyl}thiazol-2-yl)acetamide

To a suspension of N-{[(2-{4-[2-(2-acetylaminothiazol-4-yl)ethyl]phenyl}ethyl)amino]carbonothioyl}benzamide (140 mg) obtained in Production Example 23 in ethanol (1.5 ml) was added dropwise 6N aqueous sodium hydroxide solution (154.7 μl) at 0° C. The reaction mixture was stirred at 20° C. for 2 hours and neutralized with 1N hydrochloric acid at 0° C. The precipitate was collected by filtration to give N-(4-{2-[4-(2-thioureidoethyl)phenyl]ethyl}thiazol-2-yl)acetamide (98.6 mg) as a pale yellow solid.

¹H-NMR (DMSO-d₆), δ (ppm): 2.11 (3H, s), 2.68-2.79 (2H, m), 2.82-2.95 (4H, m), 3.12-3.65 (2H, m), 6.74 (1H, s), 6.96 (2H, brs), 7.14 (4H, s), 7.46-7.71 (1H, m), 12.08 (1H, s).

MS: 349.1 (M+H)+, 371.2 (M+Na)+

### Production Example 25

### Synthesis of N-[4-(2-{4-[2-(thiazol-2-ylamino)ethyl]phenyl}ethyl)thiazol-2-yl]acetamide

To a suspension of N-(4-{2-[4-(2-thioureidoethyl)phenyl]ethyl}thiazol-2-yl)acetamide (36.5 mg) obtained in Production Example 24 in isopropanol (0.3 ml) was added bromoacetaldehyde diethyl acetal (17.7 μl), and the mixture was refluxed for 24 hours. The residue was purified by preparative silica gel thin-layer chromatography with chloroform/methanol (20:1) as an eluent to give crude oil. The oil was recrystallized from an ethyl acetate/diethyl ether to give N-[4-(2-{4-[2-(thiazol-2-ylamino)ethyl]phenyl}ethyl)-thiazol-2-yl]acetamide (5.8 mg) as a white solid.

¹H-NMR (DMSO-d₆), δ (ppm): 2.11 (3H, s), 2.75-2.93 (6H, m), 3.29-3.43 (2H, m), 6.59 (1H, d, J=3.6 Hz), 6.73 (1H, s), 7.02 (1H, d, J=3.7 Hz), 7.09-7.17 (4H, m), 7.6 (1H, t, J=5.5 Hz), 12.08 (1H, brs).

MS: 373.1 (M+H)+

### Production Example 26

### Synthesis of N-[4-(2-{4-[(3-amino-1-pyrrolidinyl)methyl]phenyl}ethyl)thiazol-2-yl]acetamide dihydrochloride

Step 1

tert-Butyl (1-{4-[2-(2-acetylaminothiazol-4-yl)ethyl]benzyl}-3-pyrrolidinyl)carbamate was prepared from the compound of Step 4 of Production Example 11 in a manner similar to Step 5 of Production Example 11.

mp. 131-132.5° C.

¹H-NMR (DMSO-d₆), δ (ppm): 1.36 (9H, s), 1.44-1.68 (1H, m), 1.89-2.24 (2H, m), 2.11 (3H, s), 2.37-2.50 (2H, m), 2.61-2.74 (1H, m), 2.88 (4H, s), 3.48 (2H, s), 3.78-3.98 (1H, m), 6.73 (1H, s), 6.95 (1H, d, J=6.0 Hz), 7.13 (2H, d, J=8.0 Hz), 7.19 (2H, d, J=8.0 Hz), 12.06 (1H, brs).

MS: 445 (M+H)+

Step 2

N-[4-(2-{4-[(3-amino-1-pyrrolidinyl)methyl]phenyl}ethyl)-thiazol-2-yl]acetamide dihydrochloride was prepared from the compound of Step 1 of this Production Example in a manner similar to Step 5 of Production Example 2.

¹H-NMR (DMSO-d₆), δ (ppm): 1.91-2.37 (2H, m), 2.12 (3H, s), 2.82-3.03 (4H, m), 3.08-4.25 (5H, m), 4.33-4.49 (2H, m), 6.75 (1H, s), 7.29 (2H, d, J=8.0 Hz), 7.53 (2H, d, J=8.0 Hz), 8.55 (2H, brs), 8.69 (2H, brs), 12.10 (1H, s).

MS: 345 (M+H)+ free

### Production Example 27

### Synthesis of N-[4-(2-{4-[(3-pyrrolidinylamino)methyl]phenyl}ethyl)thiazol-2-yl]acetamide dihydrochloride

Step 1

tert-Butyl 3-({4-[2-(2-acetylaminothiazol-4-yl)ethyl]benzyl}amino)-1-pyrrolidinecarboxylate was prepared from the compound of Step 4 of Production Example 11 in a manner similar to Step 5 of Production Example 11.

¹H-NMR (DMSO-d₆), δ (ppm): 1.38 (9H, s), 1.56-1.97 (2H, m), 2.11 (3H, s), 2.88 (4H, m), 2.94-3.38 (7H, m), 3.63 (1H, brs), 6.72 (1H, s), 7.12 (2H, d, J=8.0 Hz), 7.22 (2H, d, J=8.0 Hz), 12.07 (1H, brs).

MS: 445 (M+H)+

Step 2

N-[4-(2-{4-[(3-Pyrrolidinylamino)methyl]phenyl}-ethyl)thiazol-2-yl]acetamide dihydrochloride was prepared from the compound of Step 1 of this Production Example in a manner similar to Step 5 of Production Example 2.

¹H-NMR (DMSO-d₆), δ (ppm): 1.80-2.39 (2H, m), 2.12 (3H, s), 2.77-3.95 (9H, m), 4.10-4.23 (2H, m), 6.73 (1H, s), 7.27 (2H, d, J=8.0 Hz), 7.49 (2H, d, J=8.0 Hz), 9.43 (2H, brs), 9.84 (2H, brs), 12.08 (1H, s).

MS: 345 (M+H)+ free

### Production Example 28

### Synthesis of N-[4-(2-{4-[(3-amino-1-pyrrolidinyl)ethyl]phenyl}ethyl)thiazol-2-yl]acetamide dihydrochloride

Step 1

tert-Butyl [1-(2-{4-[2-(2-acetylaminothiazol-4-yl)ethyl]phenyl}ethyl)-3-pyrrolidinyl]carbamate was prepared from the compound of Step 3 of Production Example 10 in a manner similar to Step 5 of Production Example 11.

¹H-NMR (DMSO-d₆), δ (ppm): 1.37 (9H, s), 1.45-1.63 (1H, m), 1.88-2.31 (2H, m), 2.11 (3H, s), 2.41-2.80 (7H, m), 2.87 (4H, s), 3.77-3.96 (1H, m), 6.73 (1H, s), 6.93 (1H, d, J=6.0 Hz), 7.10 (4H, s), 12.07 (1H, s).

MS: 459 (M+H)+

Step 2

N-[4-(2-{4-[(3-Amino-1-pyrrolidinyl)ethyl]phenyl}ethyl)thiazol-2-yl]acetamide dihydrochloride was prepared from the compound of Step 1 of this Production Example in a manner similar to Step 5 of Production Example 2.

¹H-NMR (DMSO-d₆), δ (ppm): 1.91-2.37 (2H, m), 2.12 (3H, s), 2.82-3.03 (4H, m), 3.08-4.25 (5H, m), 4.33-4.49 (2H, m), 6.75 (1H, s), 7.29 (2H, d, J=8.0 Hz), 7.53 (2H, d, J=8.0 Hz), 8.55 (2H, brs), 8.69 (2H, brs), 12.10 (1H, s).

MS: 345 (M+H)+ free

### Production Example 29

### Synthesis of 4-{2-[5-(2-{[amino(imino)methyl]amino}-2-oxoethyl)-2-thienyl]ethyl}-N-methyl-thiazole-2-carboxamide

Step 1

4-Chloromethyl-N-methyl-thiazole-2-carboxamide was prepared from the compound of Step 1 of Production Example 32 in a manner similar to Step 2 of Production Example 32.

¹H-NMR (CDCl₃), δ (ppm): 3.03 (3H, d, J=5.1 Hz), 4.68 (2H, s), 7.24 (1H, brs), 7.52 (1H, s).

MS: 213.1 (M+Na)+, 215.1 (M+2+Na)+

Step 2

[(2-Methylaminocarbonylthiazol-4-yl)methyl](triphenyl)phosphonium chloride was prepared from the compound of Step 1 of this Production Example in a manner similar to Step 1 of Production Example 3.

¹H-NMR (DMSO-d₆), δ (ppm): 2.78 (3H, d, J=4.5 Hz), 5.42 (2H, d, J=15 Hz), 7.59 (1H, d, J=3.5 Hz), 7.67-8.13 (16H, m).

MS: 417.1 (M−Cl−)+

Step 3

A mixture of methyl {5-[(E)-2-(2-methylaminocarbonylthiazol-4-yl)vinyl]-2-thienyl}acetate and methyl {5-[(Z)-2-(2-methylaminocarbonylthiazol-4-yl)vinyl]-2-thienyl}acetate (E:Z=5:1) was prepared from the compound of Step 2 of this Production Example in a manner similar to Step 3 of Production Example 30.

¹H-NMR (DMSO-d₆), δ (ppm): 2.83 (3H×⅚, d, J=4.8 Hz), 2.88 (3H×⅙, d, J=5.2 Hz), 3.64 (3H×⅙, s), 3.66 (3H×⅚, s), 3.91 (2H×⅙, s), 3.95 (2H×⅚, s), 6.44 (1H×⅙, d, J=12.4 Hz), 6.84 (1H×⅙, d, J=12.8 Hz), 6.88 (1H×⅙, d, J=3.6 Hz), 6.93 (1H×⅚, d, J=3.6 Hz), 6.96 (1H×⅚, d, J=15.8 Hz), 7.08 (1H×⅚, d, J=3.7 Hz), 7.15 (1H×⅙, d, J=3.6 Hz), 7.61 (1H×⅚, d, J=15.8 Hz), 7.88 (1H×⅚, s), 7.97 (1H×⅙, s), 8.75-8.82 (1H, m).

MS: 323.14 (M+H)+

Step 4

Methyl {5-[2-{2-methylaminocarbonylthiazol-4-yl)ethyl]-2-thienyl}acetate was prepared from the compound of Step 3 of this Production Example in a manner similar to Step 5 of Production Example 32.

¹H-NMR (DMSO-d₆), δ (ppm): 2.79 (3H, d, J=4.8 Hz), 3.04-3.11 (2H, m), 3.15-3.22 (2H, m), 3.62 (3H, s), 3.83 (2H, s), 6.69 (1H, d, J=3.3 Hz), 6.74 (1H, d, J=3.6 Hz), 7.63 (1H, s), 8.65-8.77 (1H, m).

MS: 325.16 (M+H)+

Step 5

4-{2-[5-(2-{[Amino(imino)methyl]amino}-2-oxoethyl)-2-thienyl]ethyl}-N-methyl-thiazole-2-carboxamide was prepared from the compound of Step 4 of this Production Example in a manner similar to Step 4 of Production Example 4.

¹H-NMR (DMSO-d₆), δ (ppm): 2.79 (3H, s), 3.02-3.1 (2H, m), 3.11-3.19 (2H, m), 3.5 (2H, brs), 6.62 (2H, s), 6.67 (2H, brs), 7.63 (1H, s), 7.78 (2H, brs), 8.7 (1H, brs).

MS: 352.1 (M+H)+

### Production Example 30

### Synthesis of 2-{5-[2-(2-acetylaminomethylthiazol-4-yl)ethyl]-2-thienyl}-N-[amino(imino)methyl]acetamide

Step 1

The mixture of N-(2-amino-2-thioxoethyl)acetamide (1 g) and 1,3-dichloroacetone (1.10 g) in ethanol (10 ml) was heated under reflux for 2 hours. The resulting pale brown solution was cooled to 20° C., and the solvent was removed. To the residue was added chloroform (30 ml). The mixture was washed with saturated aqueous sodium hydrogen carbonate solution (20 ml) and brine (20 ml) and dried over magnesium sulfate. After removal of the solvent, the resulting syrup (1.856 g, 120% mass balance) was purified by flash column chromatography over silica gel (ethyl acetate:chloroform=2:1) to give N-[(4-chloromethylthiazol-2-yl)methyl]acetamide (786.6 mg) as light yellow sticky oil.

¹H-NMR (CDCl₃), δ (ppm): 2.07 (3H, s), 4.66 (2H, s), 4.73 (2H, d, J=5.5 Hz), 6.36 (1H, brs).

MS: 205.10 (M+H)+, 207.09 (M+2+Na)+

Step 2

[(2-Acetylaminomethylthiazol-4-yl)methyl](triphenyl)phosphonium chloride was prepared from the compound of Step 1 of this Production Example in a manner similar to Step 1 of Production Example 3.

¹H-NMR (DMSO-d₆), δ (ppm): 1.84 (3H, s), 4.32 (2H, d, J=6 Hz), 5.33 (2H, d, J=15 Hz), 7.27 (1H, d, J=3.5 Hz), 7.65-7.95 (15H, m), 8.71 (1H, t, J=5.8 Hz).

MS: 431.2 (M−Cl−)+

Step 3

[(2-Acetylaminomethylthiazol-4-yl)methyl](triphenyl)phosphonium chloride (700 mg) and dimethylformamide (2.5 ml) were combined under nitrogen atmosphere, and then potassium tert-butoxide (185 mg) was added to the suspension at 0° C. The reaction mixture was stirred at 0° C. for 15 minutes, and methyl (5-formyl-2-thienyl)acetate (263 mg) in dimethylformamide (2.5 ml) was added to the mixture at 0° C. The reaction mixture was stirred at 25° C. for 2 hours. Water was added to the mixture. The mixture was extracted with ethyl acetate, washed with brine, dried over magnesium sulfate, and evaporated in vacuo to give crude brown oil. The crude brown oil was purified by flash column chromatography over silica gel with chloroform/methanol (30:1→20:1) as an eluent to give a mixture of methyl {5-[(E)-2-(2-acetylaminomethylthiazol-4-yl)vinyl]-2-thienyl}acetate and methyl {5-[(Z)-2-(2-acetylaminomethylthiazol-4-yl)vinyl]-2-thienyl}acetate (E:Z=5:1) (323 mg) as a yellow solid.

¹H-NMR (DMSO-d₆), δ (ppm): 1.88 (3H×⅙, s), 1.91 (3H×⅚, s), 3.64 (3H×⅙, s), 3.66 (3H×⅚, s), 3.89 (2H×⅙, s), 3.93 (2H×⅚, s), 4.41-4.63 (2H, m), 6.34 (1H×⅙, d, J=12.5 Hz), 6.67 (1H×⅙, d, J=12.5 Hz), 6.79-7.61 (3H+2H×⅚, m), 8.61-8.85 (1H, m).

MS: 337.1 (M+H)+

Step 4

A mixture of methyl {5-[(E)-2-(2-acetylaminomethylthiazol-4-yl)vinyl]-2-thienyl}acetate and methyl {5-[(Z)-2-(2-acetylaminomethylthiazol-4-yl)vinyl]-2-thienyl}acetate (E:Z=5:1; 323 mg), methanol (2 ml), tetrahydrofuran (1 ml) and 10% palladium on carbon (305 mg) were sequentially combined under nitrogen atmosphere. The mixture was stirred at 25° C. for 11 hours under hydrogen atmosphere (4 atm). The reaction mixture was filtered through a Celite pad, and the filtrate was concentrated in vacuo. The residue was purified by flash column chromatography over silica gel with chloroform/methanol (10:0→10:1) as an eluent to give methyl {5-[2-(2-acetylaminomethylthiazol-4-yl)ethyl]-2-thienyl}acetate (222 mg) as colorless oil.

¹H-NMR (CDCl₃), δ (ppm): 2.07 (3H, s), 3.05-3.22 (4H, m), 3.72 (3H, s), 3.76 (2H, s), 4.72 (2H, d, J=5.5 Hz), 6.36 (1H, brs), 6.62 (1H, d, J=3.3 Hz), 6.72 (1H, d, J=3.3 Hz), 6.85 (1H, s).

MS: 339.1 (M+H)+

Step 5

2-{5-[2-(2-Acetylaminomethylthiazol-4-yl)ethyl]-2-thienyl}-N-[amino(imino)methyl]acetamide was prepared from the compound of Step 4 of this Production Example in a manner similar to Step 4 of Production Example 4.

¹H-NMR (DMSO-d₆), δ (ppm): 1.89 (3H, s), 2.92-3.01 (2H, m), 3.03-3.14 (2H, m), 3.5 (2H, s), 4.48 (2H, d, J=4.8 Hz), 6.61 (1H, d, J=3.3 Hz), 6.63 (1H, d, J=3.6 Hz), 6.57 (2H, brs), 7.21 (1H, s), 7.79 (2H, brs), 8.71 (1H, brs).

MS: 366.24 (M+H)+

### Production Example 31

### Synthesis of N-{4-[2-(5-{[amino(imino)methyl]amino}-2-thienyl)ethyl]thiazol-2-yl}acetamide hydrochloride

Step 1

N-{4-[(E)-2-(5-Nitro-2-thienyl)vinyl]thiazol-2-yl}acetamide was prepared from 5-nitro-thiophene-2-carbaldehyde in a manner similar to Step 2 of Production Example 21.

¹H-NMR (DMSO-d₆), δ (ppm): 2.16 (3H, s), 7.31 (1H, d, J=15.4 Hz), 7.36 (1H, d, J=15.4 Hz), 7.39 (1H, d, J=4 Hz), 7.39 (1H, s), 8.1 (1H, d, J=4.4 Hz), 12.27 (1H, s).

MS: 296.0 (M+H)+, 318.1 (M+Na)+

Step 2

N-{4-[(E)-2-(5-Nitro-2-thienyl)vinyl]thiazol-2-yl}acetamide (300 mg), dimethylformamide (20 ml), methanol (2 ml), ethyl acetate (1 ml) and 10% palladium on carbon (50% wet) (108 mg) were combined under nitrogen atmosphere. The mixture was stirred at 25° C. under hydrogen atmosphere (4 atm) for 24 hours. The reaction mixture was filtered through a Celite pad, and the filtrate was concentrated in vacuo. Saturated aqueous sodium hydrogen carbonate solution was added, and the resulting precipitate was collected by filtration to give a black solid. The black solid was dissolved in chloroform/methanol (10:1), dried over magnesium sulfate, filtered, and evaporated to give crude N-{4-[(E)-2-(5-amino-2-thienyl)vinyl]thiazol-2-yl}acetamide (114 mg, MS: 266.12 (M+H)⁺) as brown amorphous. To the crude N-{4-[(E)-2-(5-amino-2-thienyl)vinyl]thiazol-2-yl}acetamide (114 mg) in tetrahydrofuran (0.1 ml) was added N,N′-bis(tert-butoxycarbonyl)-1H-pyrazole-1-carboxamidine (236 mg), and the mixture was stirred at 25° C. for 14 hours. The volatiles were evaporated, and the residue was purified by flash column chromatography over silica gel with chloroform/methanol (10:0→10:2) as an eluent to give di-tert-butyl ((E)-{5-[(E)-2-(2-acetylaminothiazol-4-yl)vinyl]-2-thienylamino}-methylidene)biscarbamate (67.4 mg, MS: 508.34 (M+H)⁺) as a red solid. Di-tert-butyl ((E)-{5-[(E)-2-(2-acetylamino-thiazol-4-yl)vinyl]-2-thienylamino}methylidene)biscarbamate (67.4 mg), methanol (1 ml), tetrahydrofuran (1 ml) and 10% palladium on carbon (50% wet, 68 mg) were combined under nitrogen atmosphere. The mixture was stirred at 25° C. under hydrogen atmosphere (4 atm) for 24 hours. The reaction mixture was filtered through a Celite pad, and the filtrate was concentrated in vacuo. The residue was purified by preparative silica gel thin-layer chromatography with chloroform/ethyl acetate (1:1) as an eluent to give di-tert-butyl ((E)-{5-[2-(2-acetylaminothiazol-4-yl)ethyl]-2-thienylamino}methylidene)-biscarbamate (7.7 mg) as pale yellow oil.

¹H-NMR (CDCl₃), δ (ppm): 1.53 (18H, s), 2.26 (3H, s), 2.92-3.14 (4H, m), 4.83 (1H, brs), 6.47 (1H, d, J=4 Hz), 6.5 (1H, d, J=3.7 Hz), 6.55 (1H, s), 10.69 (1H, brs), 11.43 (1H, s).

MS: 510.37 (M+H)+

Step 3

N-{4-[2-(5-{[Amino(imino)methyl]amino}-2-thienyl)ethyl]thiazol-2-yl}acetamide hydrochloride was prepared from the compound of Step 2 of this Production Example in a manner similar to Step 5 of Production Example 2.

¹H-NMR (CD₃OD), δ (ppm): 2.28 (3H, s), 3.06 (2H, t, J=7.3 Hz), 3.2 (2H, t, J=7.5 Hz), 6.76 (1H, d, J=3.7 Hz), 6.83 (1H, d, J=3.6 Hz), 6.89 (1H, s).

MS: 310.14 (M+H)+ free

### Production Example 32

### Synthesis of 4-{2-[5-(2-{[amino(imino)methyl]amino}-2-oxoethyl)-2-thienyl]ethyl}-N,N-dimethyl-thiazole-2-carboxamide

Step 1

To a solution of ethyl 4-chloromethylthiazole-2-carboxylate (1.386 g) in ethanol (10 ml) was added 1N aqueous sodium hydroxide solution (10 ml), and the mixture was stirred at 25° C. for 30 minutes. The pH of the reaction mixture was adjusted to 1 with 1N hydrochloric acid, and the mixture was extracted with ethyl acetate (30 ml×3). The extract was dried over magnesium sulfate and evaporated to give 4-chloromethylthiazole-2-carboxylic acid (1.197 g) as a brown solid.

¹H-NMR (DMSO-d₆), δ (ppm): 4.9 (2H, s), 8.11 (1H, s), 14.14 (1H, brs).

MS: 178.02 (M+H)+, 180.00 (M+2+Na)+

Step 2

To a solution of the compound of Step 1 of this Production Example (322.5 mg) in dichloromethane (6.5 ml) were added dimethylamine hydrochloride (148.1 mg), 1-hydroxy-1H-benzotriazole (HOBt, 245.4 mg) and 1-(3-dimethylaminopropyl)-3-ethylcarbodiimide (EDCI, 332 μl) at 0° C., and the mixture was stirred at 20° C. for 2 hours. The reaction mixture was diluted with dichloromethane (10 ml), washed with water, and saturated aqueous sodium hydrogen carbonate solution and brine. The organic layer was dried over magnesium sulfate and evaporated in vacuo to give crude brown oil. The crude oil was purified by flash column chromatography over silica gel with chloroform/ethyl acetate (10:1) as an eluent to give 4-chloromethyl-N,N-dimethylthiazole-2-carboxamide (313.9 mg) as a pale yellow solid.

¹H-NMR (CDCl₃), δ (ppm): 3.16 (3H, s), 3.6 (3H, s), 4.71 (2H, s), 7.49 (1H, s).

MS: 205.1 (M+H)+

Step 3

[(2-Dimethylaminocarbonylthiazol-4-yl)methyl](triphenyl)phosphonium chloride was prepared from the compound of Step 2 of this Production Example in a manner similar to Step 1 of Production Example 3.

¹H-NMR (DMSO-d₆), δ (ppm): 2.89 (3H, s), 2.94 (3H, s), 5.47 (2H, d, J=15.1 Hz), 7.62-7.96 (16H, m).

MS: 431.2 (M−Cl−)+

Step 4

Methyl {5-[(E)-2-(2-dimethylaminocarbonylthiazol-4-yl)vinyl]-2-thienyl}acetate was prepared from the compound of Step 3 of this Production Example in a manner similar to Step 3 of Production Example 30.

¹H-NMR (DMSO-d₆), δ (ppm): 3.06 (3H, s), 3.48 (3H×⅛, s), 3.56 (3H×⅞, s), 3.63 (3H×⅛, s), 3.66 (3H×⅞, s), 3.89 (2H×⅛, s), 3.94 (2H×⅞, s), 6.46 (1H×⅛, d, J=12.8 Hz), 6.77 (1H×⅛, d, J=12.4 Hz), 6.87 (1H×⅛, d, J=3.6 Hz), 6.91 (1H×⅞, d, J=3.6 Hz), 6.96 (1H×⅞, d, J=15.7 Hz), 7.12 (1H×⅞, d, J=3.7 Hz), 7.18 (1H×⅛, d, J=3.6 Hz), 7.49 (1H×⅞, d, J=15.7 Hz), 7.88 (1H×⅞, s), 7.95 (1H×⅛, s).

MS: 337.1 (M+H)+, 359.0 (M+Na)+

Step 5

A mixture of methyl {5-[(E)-2-(2-dimethylaminocarbonylthiazol-4-yl)vinyl]-2-thienyl}acetate and methyl {5-[(Z)-2-(2-dimethylaminocarbonylthiazol-4-yl)vinyl]-2-thienyl}acetate (E:Z=7:1) (378.2 g), methanol (2 ml) and 10% palladium on carbon (305 mg) were combined under nitrogen atmosphere. The mixture was stirred at 25° C. under hydrogen atmosphere (4 atm) for 8 hours. The reaction mixture was filtered through a Celite pad, and the filtrate was concentrated in vacuo. The residue was purified by preparative silica gel thin-layer chromatography with chloroform/ethyl acetate (10:1) as an eluent to give methyl {5-[2-(2-dimethylaminocarbonylthiazol-4-yl)ethyl]-2-thienyl}acetate (242.9 mg) as pale yellow oil.

¹H-NMR (DMSO-d₆), δ (ppm): 3.03 (3H, s), 3.05-3.21 (4H, m), 3.48 (3H, s), 3.62 (3H, s), 3.82 (2H, s), 6.68 (1H, d, J=3.3 Hz), 6.73 (1H, d, J=3.3 Hz), 7.6 (1H, s).

MS: 339.1 (M+H)+, 361.1 (M+Na)+

Step 6

4-{2-[5-(2-{[Amino(imino)methyl]amino}-2-oxoethyl)-2-thienyl]ethyl}-N,N-dimethylthiazole-2-carboxamide was prepared from the compound of Step 5 of this Production Example in a manner similar to Step 4 of Production Example 4.

¹H-NMR (DMSO-d₆), δ (ppm): 2.99-3.18 (7H, m), 3.48 (3H, s), 3.49 (2H, s), 6.56 (2H, brs), 6.61 (2H, s), 7.6 (1H, s), 7.79 (2H, brs).

MS: 366.1 (M+H)+

### Production Example 33

### Synthesis of 2-{5-[2-(2-acetylaminothiazol-4-yl)ethyl]-1,3,4-oxadiazol-2-yl}-N-[amino(imino)methyl]acetamide

Step 1

Methyl (2E)-3-(2-acetylaminothiazol-4-yl)acrylate was prepared from the compound of Step 4 of Reference Example in a manner similar to Step 1 of Production Example 1.

¹H-NMR (DMSO-d₆), δ (ppm): 2.15 (3H, s), 3.71 (3H×¼, s), 3.72 (3H×¾, s), 6.02 (1H×¼, d, J=12.4 Hz), 6.44 (1H×¾, d, J=15.4 Hz), 6.87 (1H×¼, d, J=12.8 Hz), 7.57 (1H×¾, d, J=15.4 Hz), 7.66 (1H×¾, s), 7.94 (1H×¼, s), 12.24 (1H, brs).

MS: 227.2 (M+H)+, 249.2 (M+Na)+

Step 2

Methyl 3-(2-acetylaminothiazol-4-yl)propanoate was prepared from the compound of Step 1 of this Production Example in a manner similar to Step 3 of Production Example 1.

¹H-NMR (DMSO-d₆), δ (ppm): 2.11 (3H, s), 2.59-2.73 (2H, m), 2.78-2.92 (2H, m), 3.59 (3H, s), 6.77 (1H, s), 12.03 (1H, s).

MS: 229.1 (M+H)+, 251.2 (M+Na)+

Step 3

3-(2-Acetylaminothiazol-4-yl)propanoic acid was prepared from the compound of Step 2 of this Production Example in a manner similar to Step 4 of Production Example 1.

¹H-NMR (DMSO-d₆), δ (ppm): 2.11 (3H, s), 2.57 (2H, t, J=7.3 Hz), 2.82 (2H, t, J=7.5 Hz), 6.75 (1H, s), 12.05 (2H, brs).

MS: 215.11 (M+H)+

Step 4

To a solution of 3-(2-acetylaminothiazol-4-yl)propanoic acid (0.649 g) in dimethylformamide (8.5 ml) were added ethyl 3-hydrazino-3-oxopropanoate (0.664 g), HOBt (0.614 g) and EDCI-HCl (0.871 g), and the mixture was stirred at 25° C. for 18 hours. The reaction mixture was poured into water (65 ml), extracted with chloroform (30 ml×3), dried over magnesium sulfate, and evaporated to give a crude pale yellow solid. The crude pale yellow solid was purified by flash column chromatography over NH-silica gel with chloroform/methanol (20:0→20:1) as an eluent to give ethyl 3-{2-[3-(2-acetylaminothiazol-4-yl)propanoyl]hydrazino}-3-oxopropanoate (465.7 mg) as a white solid.

¹H-NMR (DMSO-d₆), δ (ppm): 1.18 (3H, t, J=7.1 Hz), 2.11 (3H, s), 2.44-2.52 (2H, m), 2.83 (2H, t, J=7.7 Hz), 3.28 (2H, s), 4.08 (2H, q, J=7.1 Hz), 6.77 (1H, s), 10.05 (2H, brs), 12.01 (1H, brs).

MS: 365.2 (M+Na)+

Step 5

To a suspension of ethyl 3-{2-[3-(2-acetylaminothiazol-4-yl)propanoyl]hydrazino}-3-oxopropanoate (46.3 mg) in toluene (0.5 ml) was added phosphorus oxychloride (0.189 ml), and the mixture was stirred at 100° C. for 2 hours. The reaction mixture was poured into water, extracted with ethyl acetate, washed with brine, dried over magnesium sulfate, and evaporated. The residue was purified by flash column chromatography over NH-silica gel with chloroform/methanol (20:0→20:1) as an eluent to give ethyl {5-[2-(2-acetylaminothiazol-4-yl)ethyl]-1,3,4-oxadiazol-2-yl}acetate (19.5 mg) as a white solid.

¹H-NMR (CDCl₃), δ (ppm): 1.31 (3H, t, J=7.1 Hz), 2.25 (3H, s), 3.1-3.26 (4H, m), 3.97 (2H, s), 4.25 (2H, q, J=7.2 Hz), 6.61 (1H, s).

MS: 325.1 (M+H)+, 347.2 (M+Na)+

Step 6

2-{5-[2-(2-Acetylaminothiazol-4-yl)ethyl]-1,3,4-oxadiazol-2-yl}-N-[amino(imino)methyl]acetamide was prepared from the compound of Step 5 of this Production Example in a manner similar to Step 4 of Production Example 4.

¹H-NMR (DMSO-d₆), δ (ppm): 2.11 (3H, s), 3.02 (2H, t, J=7.5 Hz), 3.16 (2H, t, J=7.3 Hz), 3.71 (2H, s), 6.68 (2H, brs), 6.82 (1H, s), 7.74 (2H, brs), 12.05 (1H, brs).

MS: 338.20 (M+H)+

### Production Example 34

### Synthesis of 2-{5-[2-(2-acetylaminothiazol-4-yl)ethyl]-1,3,4-thiadiazol-2-yl}-N-[amino(imino)methyl]acetamide

Step 1

To a suspension of ethyl 3-{2-[3-(2-acetylaminothiazol-4-yl)propanoyl]hydrazino}-3-oxopropanoate (220 mg) in tetrahydrofuran (8 ml) was added phosphorus pentasulfide (428 mg) at 25° C. The reaction mixture was stirred at same temperature for 6 hour. Then, the insoluble material was removed by filtration, and the filtrate was evaporated to give colorless oil (328.4 mg). The residue was purified by flash column chromatography over NH-silica gel with chloroform/methanol (20:0→20:1) as an eluent to give ethyl {5-[2-(2-acetylaminothiazol-4-yl)ethyl]-1,3,4-thiadiazol-2-yl}acetate (76.6 mg) as a white solid.

¹H-NMR (DMSO-d₆), δ (ppm): 1.2 (3H, t, J=7.1 Hz), 2.12 (3H, s), 3.05 (2H, t, J=7.3 Hz), 3.45 (2H, t, J=7.3 Hz), 4.13 (2H, q, J=7.1 Hz), 4.24 (2H, s), 6.83 (1H, s), 12.1 (1H, s).

MS: 341.19 (M+H)+

Step 2

2-{5-[2-(2-Acetylaminothiazol-4-yl)ethyl]-1,3,4-thiadiazol-2-yl}-N-[amino(imino)methyl]acetamide was prepared from the compound of Step 1 of this Production Example in a manner similar to Step 4 of Production Example 4.

¹H-NMR (DMSO-d₆), δ (ppm): 2.12 (3H, s), 3.04 (2H, t, J=7.5 Hz), 3.39 (2H, t, J=7.5 Hz), 3.89 (2H, s), 6.68 (2H, brs), 6.83 (1H, s), 7.78 (2H, brs), 12.06 (1H, brs).

MS: 354.16 (M+H)+

### Production Example 35

### Synthesis of 2-{5-[2-(2-acetylaminothiazol-4-yl)ethyl]-1-methyl-1H-pyrrol-2-yl}-N-[amino(imino)methyl]acetamide

Step 1

To a solution of methyl (1-methyl-1H-pyrrol-2-yl)acetate (5 g) in dimethylformamide (65 ml) was added dropwise oxalyl chloride (3.42 ml) at 0° C. over 10 minutes. After stirring for 30 minutes, to the reaction mixture was added saturated aqueous sodium hydrogen carbonate solution (150 ml) and 1N sodium hydroxide solution (150 ml), and the mixture was then extracted 3 times with ethyl acetate. The combined organic layer was washed with 1N hydrochloric acid, saturated aqueous sodium hydrogen carbonate solution and brine, dried over magnesium sulfate, and evaporated to give crude oil. The crude oil was purified by flash column chromatography over silica gel with hexane/ethyl acetate (2:1) as an eluent to give methyl (5-formyl-1-methyl-1H-pyrrol-2-yl)acetate (4.674 g) as a pale yellow solid.

¹H-NMR (CDCl₃), δ (ppm): 3.69 (2H, s), 3.74 (3H, s), 3.91 (3H, s), 6.17 (1H, d, J=4 Hz), 6.87 (1H, d, J=4 Hz), 9.5 (1H, s).

MS: 182.2 (M+H)+

Step 2

Methyl {5-[(E)-2-(2-acetylaminothiazol-4-yl)vinyl]-1-methyl-1H-pyrrol-2-yl}acetate was prepared from the compound of Step 1 of this Production Example in a manner similar to Step 2 of Production Example 1.

¹H-NMR (DMSO-d₆), δ (ppm): 2.14 (3H, s), 3.48 (3H, s), 3.63 (3H, s), 3.76 (2H, s), 5.96 (1H, d, J=3.6 Hz), 6.39 (1H, d, J=3.7 Hz), 6.8 (1H, d, J=15.8 Hz), 7 (1H, s), 7.15 (1H, d, J=15.7 Hz), 12.16 (1H, brs).

MS: 320.2 (M+H)+, 342.1 (M+Na)+

Step 3

Methyl {5-[2-(2-acetylaminothiazol-4-yl)ethyl]-1-methyl-1H-pyrrol-2-yl}acetate was prepared from the compound of Step 2 of this Production Example in a manner similar to Step 3 of Production Example 1.

¹H-NMR (DMSO-d₆), δ (ppm): 2.11 (3H, s), 2.84 (4H, s), 3.35 (3H, s), 3.61 (3H, s), 3.66 (2H, s), 5.71 (1H, d, J=3.3 Hz), 5.77 (1H, d, J=3.7 Hz), 6.81 (1H, s), 12.07 (1H, s).

MS: 322.1 (M+H)+, 344.2 (M+Na)+

Step 4

2-{5-[2-(2-Acetylaminothiazol-4-yl)ethyl]-1-methyl-1H-pyrrol-2-yl}-N-[amino(imino)methyl]acetamide was prepared from the compound of Step 3 of this Production Example in a manner similar to Step 4 of Production Example 4.

¹H-NMR (DMSO-d₆), δ (ppm): 2.11 (3H, s), 2.83 (4H, s), 3.37 (2H, s), 3.39 (3H, s), 5.64 (1H, d, J=3.6 Hz), 5.65 (1H, d, J=3.6 Hz), 6.56 (2H, brs), 6.81 (1H, s), 7.75 (2H, brs), 12.03 (1H, brs).

MS: 349.07 (M+H)+

### Production Example 36

### Synthesis of 2-{2-[2-(2-acetylaminothiazol-4-yl)ethyl]thiazol-5-yl}-N-[amino(imino)methyl]acetamide

2-{2-[2-(2-Acetylaminothiazol-4-yl)ethyl]thiazol-5-yl}-N-[amino(imino)methyl]acetamide is prepared in a manner similar to Production Example 34.

The compounds according to the present invention, which are useful as VAP-1 inhibitors, and listed in the following tables. Numbers in the tables respectively correspond to the numbers of Production Examples described above.

| No. | Structure |
|---|---|
| 1 | 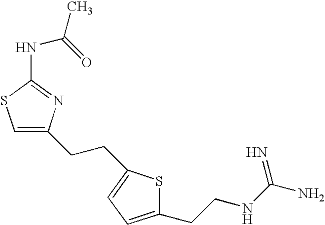 |
| 2 | 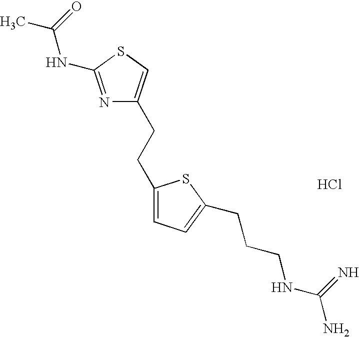 |
| 3 | 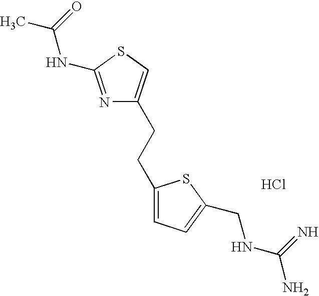 |
| 4 | 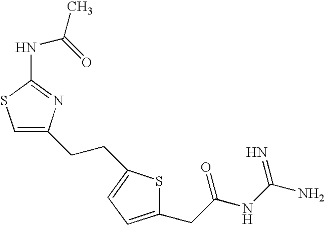 |
| 5 | 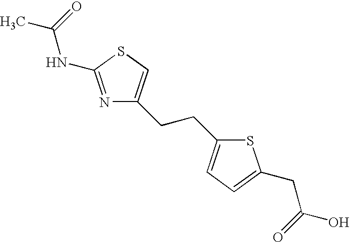 |
| 6 | 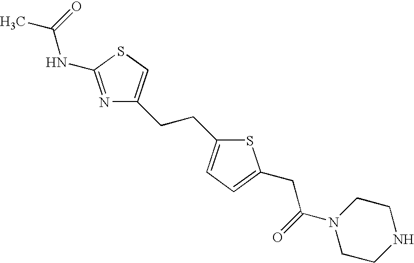 |
| 7 | 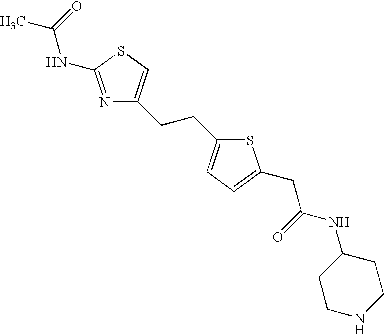 |
| 8 | 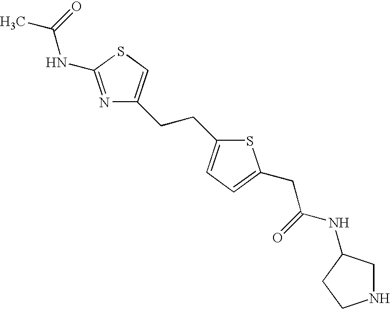 |
| 9 | 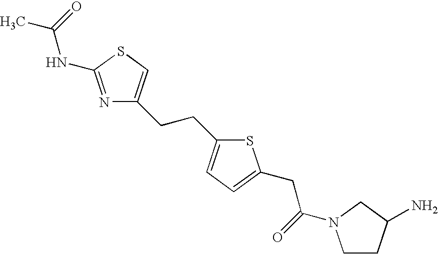 |
| 10 | 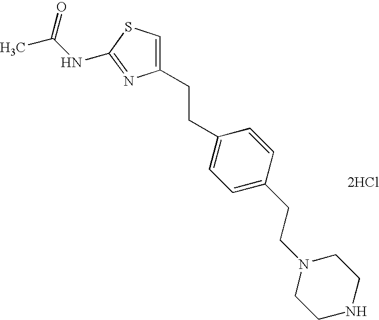 |
| 11 | 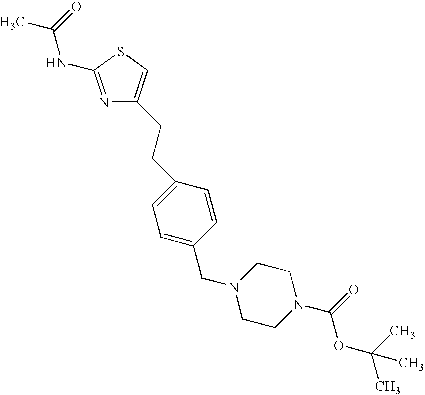 |
| 12 | 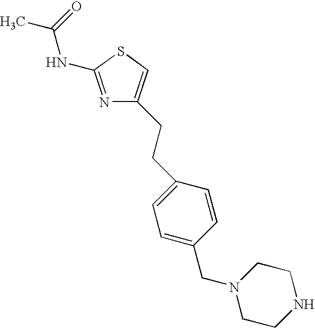 |
| 13 | 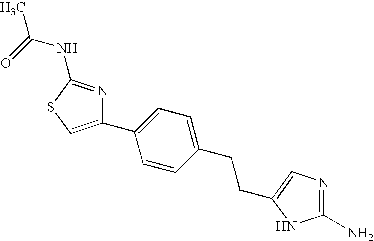 |
| 14 | 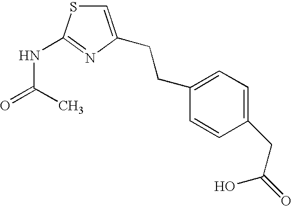 |
| 15 | 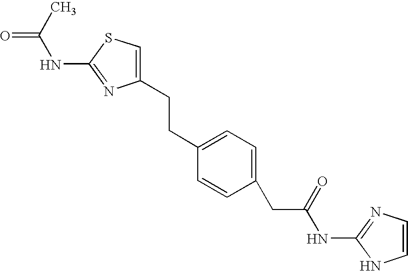 |
| 16 | 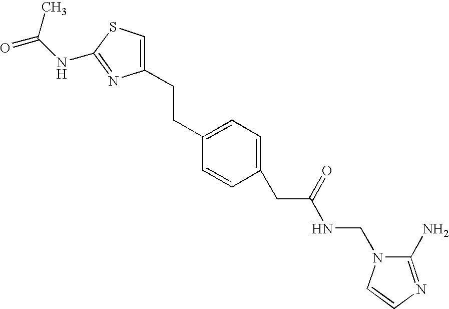 |
| 17 | 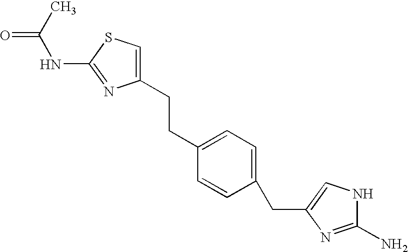 |
| 18 | 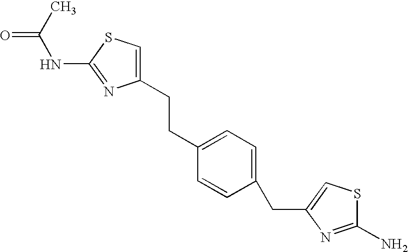 |
| 19 | 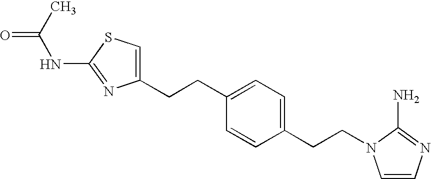 |
| 20 | 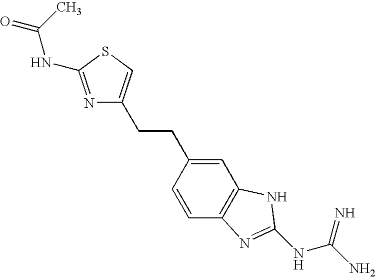 |
| 21 | 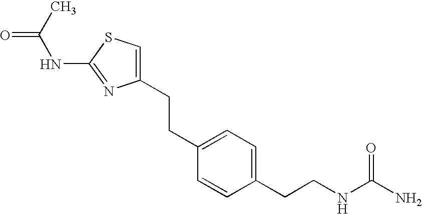 |
| 22 | 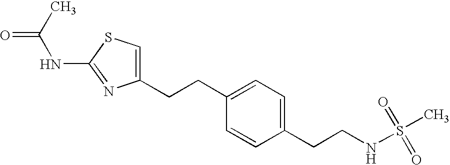 |
| 23 | 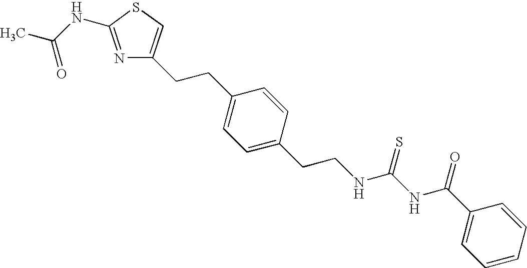 |
| 24 | 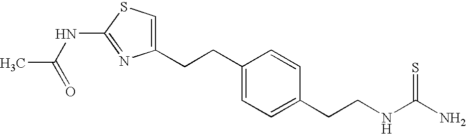 |
| 25 | 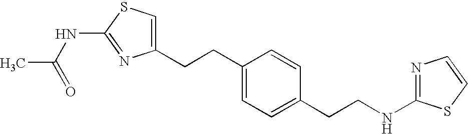 |
| 26 | 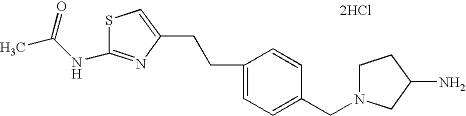 |
| 27 | 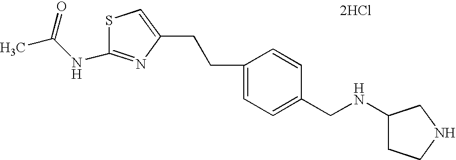 |
| 28 | 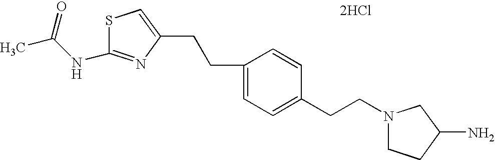 |
| 29 | 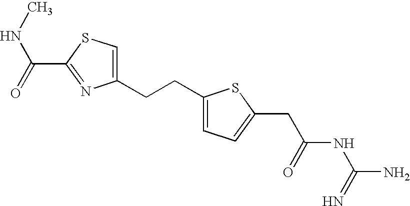 |
| 30 | 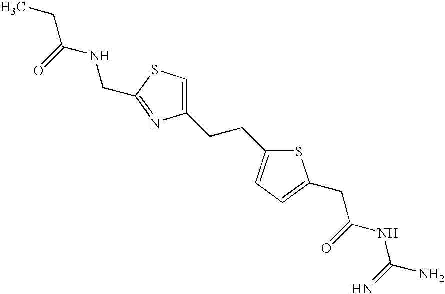 |
| 31 | 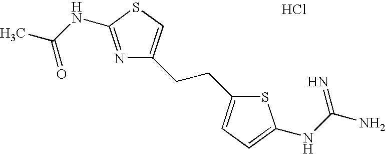 |
| 32 | 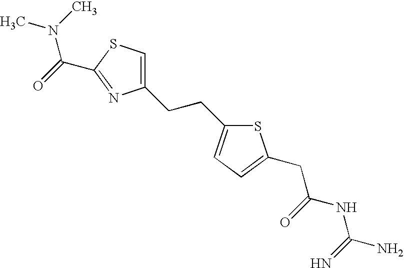 |
| 33 | 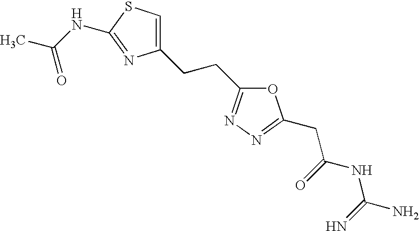 |
| 34 | 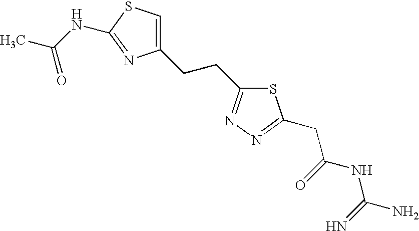 |
| 35 | 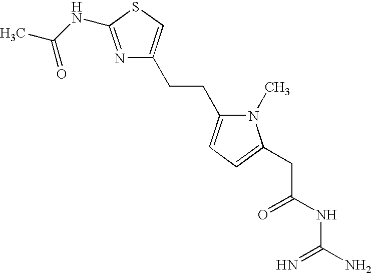 |
| 36 | 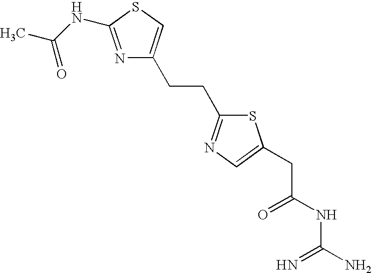 |

### Example

Inhibitory Effect of the Present Compounds on VAP-1 Enzyme (SSAO) Activity in Human Plasma.

VAP-1 enzyme (SSAO) activity in human plasma was determined by a radiochemical-enzyme assay using ¹⁴C-benzylamine as an artificial substrate. The enzyme suspension prepared from blood plasma was pre-incubated with one of the present compounds or control compound (Reference Example) in 96-well microplate at room temperature for 30 minutes. The enzyme suspension was then incubated with ¹⁴C-benzylamine (2×10⁻⁵ mol/l final concentration) in a final volume of 50 μl at 37° C. for 1 hour. The enzyme reaction was terminated by adding 2 mol/l (50 μl) citric acid. The oxidized products were directly extracted into a 200 μl toluene scintillator, and its radioactivity was measured by a scintillation spectrometer. Inhibition activity was expressed as IC₅₀ (μmol/l) value.

The present compounds inhibited the enzyme activity of human plasma SSAO in comparison with control compound as shown in Table 1.

TABLE 1
Inhibitory effect (IC₅₀ values, μM) of the present
compounds and control compound on human plasma SSAO

| Compounds | IC₅₀ values, μM |
|---|---|
| Reference Example (control) | 0.033 |
| Production Example 3 | 0.012 |
| Production Example 4 | 0.0024 |
| Production Example 17 | 0.0053 |

### INDUSTRIAL APPLICABILITY

The present invention provides a compound of the formula (I):
U-V-W-X-Y-Z (I)
wherein each symbol is as defined above, or a pharmaceutically acceptable salt thereof useful as a VAP-1 inhibitor as well as a pharmaceutical composition and a method for preventing or treating a VAP-1 associated disease, especially macular edema such as diabetic macular edema and non-diabetic macular edema, which method comprises administering to a patient in need thereof a VAP-1 inhibitor in an amount sufficient to treat the patient suffering from the VAP-1 associated disease, and the like.

This application is based on a provisional patent application No. 2004905183 filed in Australia, the contents of which are all hereby incorporated by reference.

1. A compound of the formula (I):
U-V-W-X-Y-Z (I)
wherein
U is lower alkyl;
V is —CONH— or —NR¹CO— wherein R¹ is a hydrogen or lower alkyl;
W is a bond or lower alkylene;
X is a bivalent residue derived from optionally substituted thiazole;
Y is a bond or lower alkylene; and
Z is a group of the formula: 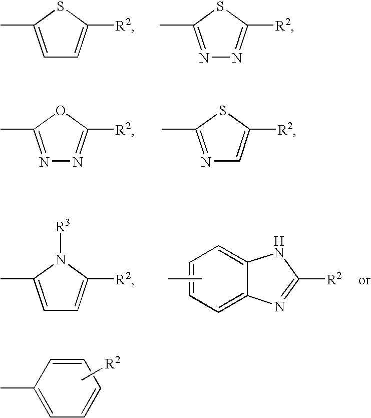
wherein R² is a group of the formula: -A-B-D-E-F-G
wherein
A is a bond or lower alkylene;
B is a bond, —NH— or 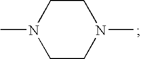
D is a bond, —CS— or —CO—;
E is a bond or —NH—;
F is a bond, —CO—, —O— or —SO₂—; and
G is lower alkyl, optionally protected amino, —OH, phenyl, 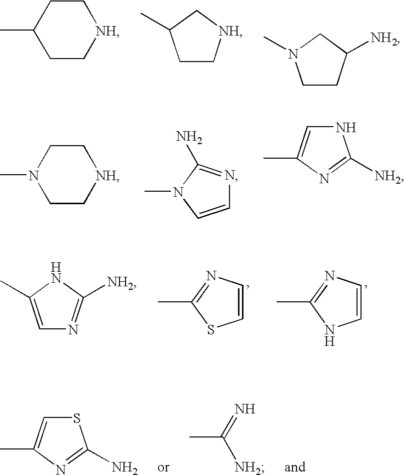
R³ is lower alkyl,
provided that
when Z is a group of the formula: 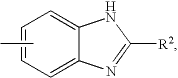
then G should not be amino,
when Z is a group of the formula: 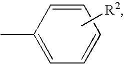
then G should not be 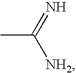
when Z is a group of the formula: 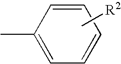
and G is optionally protected amino,
then D should be —CS—, or
then A should be lower alkylene,
B or E should be —NH— and F should be —CO—;
or a pharmaceutically acceptable salt thereof.

2. The compound of claim 1, wherein the compound is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide,
or a pharmaceutically acceptable salt thereof.

3. The compound of claim 1 or a pharmaceutically acceptable salt thereof for use as a medicament.

4. A pharmaceutical composition, which comprises, as an active ingredient, the compound of claim 1 or a pharmaceutically acceptable salt thereof.

5. The pharmaceutical composition of claim 4, wherein the compound of the formula (I) is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide.

6. Use of the compound of claim 1 or a pharmaceutically acceptable salt thereof for preparing a medicament as a VAP-1 inhibitor.

7. The use of claim 6, wherein the compound is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide,
or a pharmaceutically acceptable salt thereof.

8. Use of the compound of claim 1 or a pharmaceutically acceptable salt thereof for preparing a medicament for the prophylaxis or treatment of a VAP-1 associated disease.

9. The use of claim 8, wherein said VAP-1 associated disease is selected from the group consisting of cirrhosis, essential stabilized hypertension, diabetes, arthrosis, endothelium damage (in diabetes, atherosclerosis and hypertension), a cardiovascular disorder associated with diabetes and uremia, pain associated with gout and arthritis, retinopathy (in diabetes patients), a (connective tissue) inflammatory disease or condition (rheumatoid arthritis, ankylosing spondylitis, psoriatic arthritis, osteoarthritis, degenerative joint disease, Reiter's syndrome, Sjögren's syndrome, Behçet's syndrome, relapsing polychondritis, systemic lupus erythematosus, discoid lupus erythematosus, systemic sclerosis, eosinophilic fasciitis, polymyositis, dermatomyositis, polymyalgia rheumatica, vasculitis, temporal arteritis, polyarteritis nodosa, Wegener's granulomatosis, mixed connective tissue disease, and juvenile rheumatoid arthritis), a gastrointestinal inflammatory disease or condition [Crohn's disease, ulcerative colitis, irritable bowel syndrome (spastic colon), fibrotic conditions of the liver, inflammation of the oral mucosa (stomatitis), and recurrent aphtous stomatitis], a central nervous system inflammatory disease or condition (multiple sclerosis, Alzheimer's disease, and ischemia-reperfusion injury associated with ischemic stroke), a pulmonary inflammatory disease or condition (asthma, adult respiratory distress syndrome, and chronic obstructive pulmonary disease), a (chronic) skin inflammatory disease or condition (psoriasis, allergic lesions, lichen planus, pityriasis rosea, contact dermatitis, atopic dermatitis, and pityriasis rubra pilaris), a disease related to carbohydrate metabolism (diabetes and complications from diabetes) including microvascular and macrovascular disease (atherosclerosis, vascular retinopathies, retinopathy, nephropathy, nephrotic syndrome and neuropathy (polyneuropathy, mononeuropathies and autonomic neuropathy), foot ulcers, joint problems, and increased risk of infection), a disease related to aberrations in adipocyte differentiation or function or smooth muscle cell function (atherosclerosis and obesity), a vascular disease [atheromatous ateriosclerosis, nonatheromatous ateriosclerosis, ischemic heart disease including myocardial infarction and peripheral arterial occlusion, Raynaud's disease and phenomenon, and thromboangiitis obliterans (Buerger's disease)], chronic arthritis, inflammatory bowel diseases, skin dermatoses, diabetes mellitus, SSAO-mediated complication [diabetes (insulin dependent diabetes mellitus (IDDM) and non-insulin dependent diabetes mellitus (NIDDM)) and vascular complication (heart attack, angina, strokes, amputations, blindness, and renal failure)], macular edema (diabetic and non-diabetic macular edema), hepatitis, and transplantation.

10. The use of claim 9, wherein said VAP-1 associated disease is macular edema.

11. The use of claim 10, wherein said macular edema is diabetic macular edema.

12. The use of claim 10, wherein said macular edema is non-diabetic macular edema.

13. A VAP-1 inhibitor, which comprises the compound of claim 1 or a pharmaceutically acceptable salt thereof.

14. A method for preventing or treating macular edema, which method comprises administering to a subject in need thereof a VAP-1 inhibitor in an amount sufficient to treat said subject for macular edema.

15. The method of claim 14, wherein the VAP-1 inhibitor is
N-(4-{2-[5-({[amino(imino)methyl]amino}methyl)-2-thienyl]ethyl}thiazol-2-yl)acetamide,
2-{5-[2-(2-acetylaminothiazol-4-yl)ethyl]-2-thienyl}-N-[amino(imino)methyl]acetamide or
N-[4-(2-{4-[(2-amino-1H-imidazol-4-yl)methyl]phenyl}ethyl)thiazol-2-yl]acetamide,
or a pharmaceutically acceptable salt thereof.

16. A method for preventing or treating a VAP-1 associated disease, which method comprises administering an effective amount of the compound of claim 1 or a pharmaceutically acceptable salt thereof to a subject in need thereof.

17. The method of claim 16, wherein said VAP-1 associated disease is selected from the group consisting of cirrhosis, essential stabilized hypertension, diabetes, arthrosis, endothelium damage (in diabetes, atherosclerosis and hypertension), a cardiovascular disorder associated with diabetes and uremia, pain associated with gout and arthritis, retinopathy (in diabetes patients), a (connective tissue) inflammatory disease or condition (rheumatoid arthritis, ankylosing spondylitis, psoriatic arthritis, osteoarthritis, degenerative joint disease, Reiter's syndrome, Sjögren's syndrome, Behçet's syndrome, relapsing polychondritis, systemic lupus erythematosus, discoid lupus erythematosus, systemic sclerosis, eosinophilic fasciitis, polymyositis, dermatomyositis, polymyalgia rheumatica, vasculitis, temporal arteritis, polyarteritis nodosa, Wegener's granulomatosis, mixed connective tissue disease, and juvenile rheumatoid arthritis), a gastrointestinal inflammatory disease or condition [Crohn's disease, ulcerative colitis, irritable bowel syndrome (spastic colon), fibrotic conditions of the liver, inflammation of the oral mucosa (stomatitis), and recurrent aphtous stomatitis], a central nervous system inflammatory disease or condition (multiple sclerosis, Alzheimer's disease, and ischemia-reperfusion injury associated with ischemic stroke), a pulmonary inflammatory disease or condition (asthma, adult respiratory distress syndrome, and chronic obstructive pulmonary disease), a (chronic) skin inflammatory disease or condition (psoriasis, allergic lesions, lichen planus, pityriasis rosea, contact dermatitis, atopic dermatitis, and pityriasis rubra pilaris), a disease related to carbohydrate metabolism (diabetes and complications from diabetes) including microvascular and macrovascular disease (atherosclerosis, vascular retinopathies, retinopathy, nephropathy, nephrotic syndrome and neuropathy (polyneuropathy, mononeuropathies and autonomic neuropathy), foot ulcers, joint problems, and increased risk of infection), a disease related to aberrations in adipocyte differentiation or function or smooth muscle cell function (atherosclerosis and obesity), a vascular disease [atheromatous ateriosclerosis, nonatheromatous ateriosclerosis, ischemic heart disease including myocardial infarction and peripheral arterial occlusion, Raynaud's disease and phenomenon, and thromboangiitis obliterans (Buerger's disease)], chronic arthritis, inflammatory bowel diseases, skin dermatoses, diabetes mellitus, SSAO-mediated complication [diabetes (insulin dependent diabetes mellitus (IDDM) and non-insulin dependent diabetes mellitus (NIDDM)) and vascular complication (heart attack, angina, strokes, amputations, blindness and renal failure)], macular edema (diabetic and non-diabetic macular edema), hepatitis, and transplantation.

18. The method of claim 17, wherein said VAP-1 associated disease is macular edema.

19. The method of claim 18, wherein said macular edema is diabetic macular edema.

20. The method of claim 18, wherein said macular edema is non-diabetic macular edema.
